(19) United States
(12) Patent Application Publication
(10) Pub. No.: US 2025/0373077 A1
(43) Pub. Date: Dec. 4, 2025

(54) Advanced Magnetic Metamaterial Networks for Spatially-Engineered Magnetoinductive Waves for Signal Transport

(71) Applicant: The Regents of the University of California, Oakland, CA (US)

(72) Inventors: Peter Tseng, Irvine, CA (US); Amirhossein Hajiaghajani, Irvine, CA (US)

(21) Appl. No.: 18/872,814

(22) PCT Filed: Jun. 1, 2023
(86) PCT No.: PCT/US2023/024110

§ 371 Date: Dec. 7, 2024

Related U.S. Application Data
(60) Provisional application No. 63/350,517, filed on Jun. 9, 2022.

(51) Int. Cl. H02J 50/10; H02J 50/20; H04B 5/79

(52) U.S. Cl. CPC H02J 50/10; H02J 50/20; H04B 5/79

(57) ABSTRACT

Advanced magnetic metamaterial networks for spatially-engineered magnetoinductive waves for signal transport in accordance with embodiments of the invention are disclosed. In one embodiment, a metamaterial network is provided, the metamaterial network comprising: at least one of magneto-inductive (MI) array; wherein the at least one MI array comprises a plurality of magnetically coupled resonators configured to propagate MI surface waves; and wherein the plurality of magnetically coupled resonators creates a magnetic metamaterial path for wireless communication using the MI surface waves.

### CROSS-REFERENCE TO RELATED APPLICATION

The current application claims priority to U.S. Provisional Patent Application No. 63/350,517 filed on Jun. 9, 2022, the disclosure of which is incorporated herein by reference.

### FEDERAL FUNDING SUPPORT

This invention was made with Government support under Grant No. ECCS-1942364, awarded by the National Science Foundation. The Government has certain rights in the invention.

### FIELD OF THE INVENTION

The present invention generally relates to wireless communications and more specifically to advanced magnetic metamaterial networks for spatially-engineered magnetoinductive waves for wireless signal and power transport.

### BACKGROUND

Radio-Frequency (RF) magnetic fields be utilized in modern biomedical imaging, sensing, and therapeutic technologies. These may include functions, such as where RF fields are exchanged with patients to create critical magnetic resonance images or power/monitor implanted devices (e.g., glucose monitors), as well as within emerging technologies that seek to remotely control drug release, induce apoptosis in cancer cells, manipulate neuronal circuits in the body, and more. RF magnetic fields also can be utilized in signal and power transfer to moving objects (e.g., electric vehicles, robots).

RF magnetic fields are unique to other physical phenomena in their minimal interaction with biological systems. While optical/electric fields are heavily absorbed/scattered by skin and tissue, magnetic fields can penetrate relatively deeply into tissue with minimal perturbation. Typically, RF magnetic fields are only absorbed indirectly through eddy currents which may minimize specific absorption rate (SAR), while maximizing penetration depth. For example, during magnetic resonance imaging MRI, several 100 s of watts of RF magnetic power can be applied to small regions without exceeding SAR safe limits.

### SUMMARY OF THE INVENTION

The various embodiments of the present advance magnetic metamaterial networks for spatially-engineered magnetoinductive waves (may also be referred to as “magnetic metamaterials” or “magnetic metamaterial networks” or “metamaterial networks”) contain several features, no single one of which is solely responsible for their desirable attributes. Without limiting the scope of the present embodiments, their more prominent features will now be discussed below. After considering this discussion, and particularly after reading the section entitled “Detailed Description,” one will understand how the features of the present embodiments provide the advantages described here.

In an embodiment of a first aspect, a metamaterial network is provided, the metamaterial network comprising: at least one of magneto-inductive (MI) array; wherein the at least one MI array comprises a plurality of magnetically coupled resonators configured to propagate MI surface waves; and wherein the plurality of magnetically coupled resonators creates a magnetic metamaterial path for wireless communication using the MI surface waves.

In another embodiment of the first aspect, the at least one MI array includes a surface-spread MI array.

In another embodiment of the first aspect, the surface-spread MI array comprises a nested colony of small to large resonators with a same resonant frequency.

In another embodiment of the first aspect, the surface-spread MI array comprises a two-dimensional array of identical resonators.

In another embodiment of the first aspect, the surface-spread MI array comprises a hybrid arrangement.

In another embodiment of the first aspect, the metamaterial network further comprises at least one peripheral device configured to monitor at least one biological signal.

In another embodiment of the first aspect, the at least one MI array is integrated into clothing.

In another embodiment of the first aspect, the at least one MI array is attached to a user's skin.

In another embodiment of the first aspect, the plurality of magnetically coupled resonators includes two neighboring resonators formed into a serpentine shape enhancing stretchability and flexibility of the at least one MI array.

In another embodiment of the first aspect, the plurality of magnetically coupled resonators includes two resonators connected via jumper wires.

In an embodiment of a second aspect, a metamaterial network is provided, the metamaterial comprising: a plurality of magneto-inductive (MI) arrays; wherein each of the plurality of MI arrays comprises magnetically coupled resonators configured to propagate MI surface waves; wherein the magnetically coupled resonators creates a magnetic metamaterial path for wireless communication using the MI surface waves; and wherein the plurality of MI arrays operates at a plurality of resonance modes.

In another embodiment of the second aspect, wherein each resonant mode's frequency is tuned using at least one lumped capacitor integrated into positions on a multiturn loop trace.

In another embodiment of the second aspect, the plurality of resonance modes includes a first mode tuned to a Wireless Power Transfer (WPT) frequency band.

In another embodiment of the second aspect, the plurality of resonance modes includes a second mode tuned to a Near-field Communication (NFC) frequency band.

In an embodiment of a third aspect, a tunable multiband skyrmion network is provided, the tunable multiband skyrmion network comprising: at least one MI array; at least one transmitter coil powered by a local source connected to at least one distributed energy source; at least one transceiver coil connected to at least one peripheral configured to travel along the at least one MI array; and wherein the tunable multiband skyrmion network is configured for power sharing using a power frequency band and data sharing using a communication frequency band.

In another embodiment of the third aspect, the power frequency band is a WPT frequency band.

In another embodiment of the third aspect, the communication frequency band is a NFC frequency band.

In another embodiment of the third aspect, the at least one peripheral is a device having a rechargeable battery.

In another embodiment of the third aspect, the tunable multiband skyrmion network further comprises a power divider and plurality of power path switches controlled by a signal path.

In another embodiment of the third aspect, wherein power safety is managed though the plurality of power path switches, wherein each of the plurality of power path switches is configured to switch off power transmission in case of detected failure.

In another embodiment of the third aspect, the at least one MI array comprises at least two resonators connected via jumper wires to create a radiation-free blind spot to enhance security, horizontal range, and allow for simple switching between two side of the at least one MI array.

### BRIEF DESCRIPTION OF THE DRAWINGS

The various embodiments of the present magnetic metamaterial networks now will be discussed in detail with an emphasis on highlighting the advantageous features. These embodiments depict the novel and non-obvious features of magnetic metamaterial networks shown in the accompanying drawings, which are for illustrative purposes only. These drawings include the following figures:

FIG. 1 is a diagram illustrating applications of RF magnetic field in biomedical technologies in accordance with an embodiment of the invention.

FIG. 2 is a diagram illustrating implementations of magnetic metamaterials in functional biomedical technologies in accordance with an embodiment of the invention.

FIGS. 3a-g are diagrams illustrating magnetic metamaterial integrated textiles enabling on-body transfer of nearfield signal power in accordance with an embodiment of the invention.

FIGS. 4a-d are diagrams illustrating in vivo multi-transponder and multi-BAN communication by textile-integrated waveguides in accordance with an embodiment of the invention.

FIGS. 5a-d are diagrams illustrating advanced magnetic metamaterials in accordance with an embodiment of the invention.

FIGS. 6a-e are diagrams illustrating magnetoinductive elements for MI wave manipulation in accordance with an embodiment of the invention.

FIGS. 7a-b are diagrams illustrating construction of magnetic metamaterials with enhanced electronic properties and programmable thermal dissipation in accordance with an embodiment of the invention.

FIG. 8 is a diagram illustrating experimental validation of biomagnetic control in advanced magnetic metamaterials in accordance with an embodiment of the invention.

FIGS. 9a-c are diagrams illustrating surface-spread magneto-inductive arrays in accordance with an embodiment of the invention.

FIGS. 10a-b are diagrams illustrating wearable and epidermal variations of magneto-inductive metamaterials for human to peripherals for communication and powering applications in accordance with an embodiment of the invention.

FIGS. 11a-d are diagrams illustrating a hybrid wired connection between neighbor resonators formed into a serpentine shape to enhance the stretchability and flexibility of the array particularly on the joints with frequent bends in accordance with an embodiment of the invention.

FIG. 12 is a diagram illustrating epidermal magneto-inductive metamaterials transferred onto human skin, tuned at NFC frequency in accordance with an embodiment of the invention.

FIG. 13 is a diagram illustrating electrical reliability of cold-pressed capacitor joints for solder-free contact to copper traces in accordance with an embodiment of the invention.

FIG. 14 is a diagram illustrating epidermal magneto-inductive metamaterial fabrication and skin transferring process in accordance with an embodiment of the invention.

FIG. 15 is a diagram illustrating hybrid connection of two resonators with flexible jumper wires allows to create radiation-free blind spot and lower the magnetoinductive waveguide loss per unit of length in accordance with an embodiment of the invention.

FIGS. 16a-b are diagrams illustrating demonstration of the fundamental and higher orders of resonance in the same multiturn loop, known as a “skyrmion” in accordance with an embodiment of the invention.

FIG. 17 is a diagram illustrating that one or more number of capacitor(s) may be placed at various positions on the multiturn loop to tune each resonance frequency in accordance with an embodiment of the invention.

FIGS. 18a-c are diagrams illustrating termination of magneto-inductive arrays using low quality resonators at the array's end in accordance with an embodiment of the invention.

FIGS. 19a-b are diagrams illustrating a tunable multiband skyrmion array of magneto-inductive metamaterials can create a low-loss network between several distributed energy sources and electric robot/vehicles close to the network in accordance with an embodiment of the invention.

FIGS. 20a-c are diagrams illustrating transmission profile of a dual band skyrmion array of magneto-inductive metamaterials in accordance with an embodiment of the invention.

FIG. 21 is a diagram illustrating transmission profile fluctuations (demonstrated by normalized current of the n^{th} resonator) caused by multiple sources (above the 1st and 30th resonators) along a terminated magneto-inductive waveguide of 30 resonators in accordance with an embodiment of the invention.

FIG. 22 is a diagram illustrating effect of transmission fluctuation versus relative position of transmitter (Tx) and receiver (Rx) nodes (caused by unterminated magneto-inductive arrays) in accordance with an embodiment of the invention.

FIG. 23 is a diagram illustrating hybrid wired connection between two neighbor resonators in a dual band magneto-inductive array tuned at 6.78 MHz and 13.56 MHz for multipurpose powering and communication applications in accordance with an embodiment of the invention.

### DETAILED DESCRIPTION OF THE DRAWINGS

The following detailed description describes the present embodiments with reference to the drawings. In the drawings, reference numbers label elements of the present embodiments. These reference numbers are reproduced below in connection with the discussion of the corresponding drawing features.

One aspect of the present embodiments includes the realization that RF technologies carry diverse requirements in RF intensities of power, magnetic field magnitude, or operating frequency. For example, MRI systems may utilize RF between 1 and 300 MHz, while driving power up to 100 s of watts. Implantable devices currently utilize nearfield communication (NFC, 13.56 MHz), while wearable devices are commonly powered using a variety of standardized power transfer protocols (300 MHz, 6.78 MHz). Further, RF-triggered drug release has emerged as a promising modality in nanomedicine, where mechanical oscillations (below 10 kHz) or nanomagnetic heating (hyperthermia at 100 to 500 kHz) can open liposomes to release therapeutics. Such liposomes have additionally emerged as a candidate for neural control through the engineered release of chemogenetic modulators. Similar stimuli can additionally be utilized to induce heating in LC tanks, which when embedded inside drug-containing materials can accelerate the release of growth factors, drugs, and more. Furthermore, magnetism-triggered heating of magnetic nanoparticles, when particles are attached or nearby cells, have been found to enable genetic expression, trigger ion channels, and enable deep brain stimulation. In addition, genetic tools may be emerging that utilize RF magnetic fields, where stimulation of the protein ferritin tagged to channels/gates can activate cellular processes through 300 kHz or 180 MHz magnetic fields. Although controversial, recent corroborating studies have found this may be driven through novel oxidative pathways.

Another aspect of the present embodiments includes the realization that consistent in such above approaches may be the need to create highly controlled, spatiotemporal RF magnetic fields. In application, these fields should be distributed across living systems as required for varying applications in local drug release, cell stimulation, device powering or more. However, modern biological RF power systems are often simplistic and incapable of such complexity. Mobile RF systems deliver energy from a single coil to a single position (i.e., a Qi charger or reverse charging capabilities on a phone), while high-power systems are typically tethered directly to the immediate rigid coils/electromagnetics of bulky power amplifiers. New, lightweight, mobile/flexible techniques to deliver RF magnetic power could unlock the potential of many emerging technologies, enable on-demand, deep tissue modulation. These could enable magnetic-driven dosing on demand that track alongside the user. Low-burden, low-cost techniques could yield new, accessible cancer or magnetic cranial imaging/stimulation treatments. Non-invasive deep brain stimulation in in-vivo models (such as mice) could be improve through lightweight interfaces that could partially untether animals from power supplies. Further, such approaches could create networks of light, implantable devices all freed from bulky batteries.

Another aspect of the present embodiments includes the realization that magnetic metamaterials may be a candidate technology to facilitate the delivery of RF magnetic power. These may include arrays of inductors that can support the propagation of magnetoinductive (MI) waves along its pathway (forming a waveguide). Such MI waves exhibit comparatively low loss in attenuating media and may be utilized as an approach to spatially transport waves across soil, salt water/ocean, during MRI, and along the body. Despite its potential, many such investigations have primarily been theoretical or in small testbeds—this is because in practice inductors orient perpendicularly and are difficult to directly implant into environments. In addition, the functional distance of RF magnetic power transported by these waves is relatively low—this is because the entire waveguide is homogeneous, active, and radiates in the nearfield.

Another aspect of the present embodiments in power transfer to moving electric loads (e.g., low power electric vehicles, robots) includes the ease of extending the MI waveguides using discrete pieces of magnetically coupled resonators. This minimizes the infrastructure needed for dynamic wireless power transfer and simplifies the network expansion. The presented MI network enables distributing the power generated by local energy sources (e.g., wind, hydro, or solar power) in remote places. The dynamic wireless power transfer enabled by the proposed MI waveguides can increase the duty cycle of robotics by eliminating the charging time in which the robot/vehicle is not on service.

The MI waveguides of the present embodiments are tuned into more than one resonant frequency, dedicating each frequency band to a particular purpose. Here, the MI waveguide possesses the first passband tuned at 6.78 MHz for wireless power transfer and the second passband tuned at 13.56 MHz for nearfield communication (NFC). The dynamic wireless power transfer realized by the present embodiments employs a power flow control through the NFC band by sharing the moving electric loads' status (e.g., battery temperature, charge status, location). NFC controlled power relays are used across the MI waveguide to enable/disable power flow (at 6.78 MHz) in case of emergency situations, or to enable power transfer on-demand to particular zones of the MI waveguides. The combined information and power sharing scheme of the present embodiments enables inter-vehicle communication and power transfer on demand. In addition, once a power sharing agreement is established between two moving vehicles (one with sufficient and one with low battery charge) through the NFC band, the vehicle with sufficient battery charge may transmit power to other loads/vehicles through the frequency band tuned at 6.78 MHz.

Another aspect of the present embodiments includes integration of emitting and non-emitting zones within a MI waveguide. The non-emitting zones transmit power and information through the dedicated frequency bands but disable RF nearfield emission (create quiet zones) to increase security, mechanical flexibility or range of the MI waveguides.

Turning now to the drawings, advance magnetic metamaterial networks for spatially-engineered magnetoinductive waves (may also be referred to as “magnetic metamaterials” or “magnetic metamaterial networks”) are further described below. In many embodiments, magnetic metamaterials may include one or more planar inductive elements that exhibit unique programmability in design and architecture. In various embodiments, magnetic metamaterials network may be built on demand and optimized to application needs in shape, depth, and locality. In several embodiments, the present embodiments may utilize elements that may modulate the transmission of MI waves in various ways. For example, these may include, but are not limited to, (1) controllable regions of near-field radiative and low-loss non-radiative sections enabling signals to pass long distances without losing amplitude while maximizing effect, (2) engineered magnetic skyrmions where metamaterials may be engineered to transmit multiple, engineered frequencies, as further described below, and (3) mixed inductive components that may modulate the local penetration depth or surface uniformity of RF signal. In combination, such architectures may enable the routing of RF magnetic fields to fit the complex need of emerging networks such as, but not limited to, biomonitoring and/or bioactuation.

In many embodiments, the present embodiments may include various magneto-inductive elements including, but not limited to, hybrid jumpered wires, multiband skyrmions, and 2 dimensional arrays. In several embodiments, such magneto-inductive elements may be different wave modulating elements that may be engineered on-demand and integrated into various types of networks to create spatio-temporally engineered nearfield radiations wherever a contactless signal/power transfer is needed. Applications of RF magnetic biomedical technologies in accordance with embodiments of the invention are further described below.

### RF Magnetic Fields in Biomedical Technologies

Radio-Frequency (RF) magnetic fields may play numerous, significant roles in modern biomedical imaging, sensing, and therapeutic technologies. These include both well-established functions, such as where RF fields are exchanged with patients to create critical magnetic resonance images or power/monitor implanted devices (such as glucose monitors), as well as within emerging technologies that seek to remotely control drug release, induce apoptosis in cancer cells, manipulate neuronal circuits in the body, and more. RF magnetic fields are unique to other physical phenomena in their minimal interaction with biological systems. While optical/electric fields may be heavily absorbed/scattered by skin and tissue, magnetic fields can penetrate relatively deeply into tissue with minimal perturbation. RF magnetic fields are only absorbed indirectly through eddy currents—this minimizes specific absorption rate (SAR), while maximizing penetration depth. For example, during MRI, several 100 s of Watts of RF magnetic power can be applied to small regions without exceeding SAR safe limits.

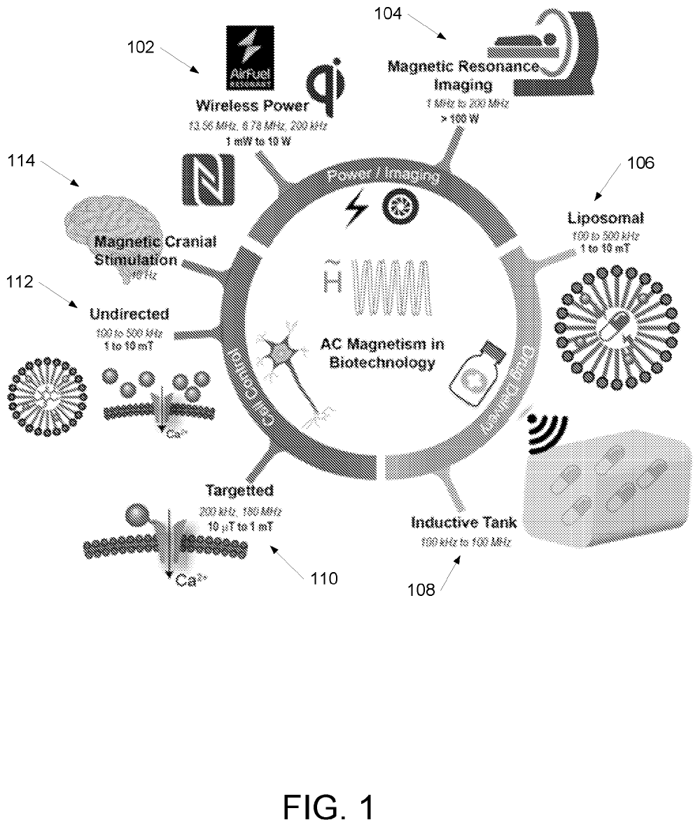

A diagram illustrating applications of RF magnetic field in biomedical technologies in accordance with an embodiment of the invention is shown in FIG. 1. These technologies carry diverse requirements in RF intensities of power, magnetic field magnitude, or operating frequency. One class of biomedical technologies that may utilize RF magnetic field applications may include power and/or imaging such as, but not limited to wireless power 102 and MRI 104 technologies. For example, MRI systems may utilize RF between 1 and 300 MHz, while driving power up to 100 s of watts. Implantable devices currently utilize nearfield communication (NFC, 13.56 MHz), while wearable devices are commonly powered using a variety of standardized power transfer protocols (300 MHz, 6.7 MHz). Another class of biomedical technologies that may utilize RF magnetic field applications may include drug delivery such as, but not limited to liposomal 106 and inductive tank 108 technologies. For example, RF-triggered drug release may be a promising modality in nanomedicine, where mechanical oscillations (below 10 kHz) or nanomagnetic heating (hyperthermia at 100 to 500 kHz) can open liposomes to release therapeutics. Such liposomes have additionally emerged as a candidate for neural control through the engineered release of chemogenetic modulators. Similar stimuli can additionally be utilized to induce heating in LC tanks, which when embedded inside drug-containing materials can accelerate the release of growth factors, drugs, and more. In addition, another class of biomedical technologies that may utilize RF magnetic field applications may include cell control such as, but not limited to targeted 110, undirected 112, and magnetic cranial stimulation 114. For example, magnetism-triggered heating of magnetic nanoparticles, when particles are attached or nearby cells, may enable genetic expression, trigger ion channels, and enable deep brain stimulation. In addition, genetic tools may utilize RF magnetic fields, where stimulation of the protein ferritin tagged to channels/gates can activate cellular processes through 300 kHz or 180 MHz magnetic fields. Although controversial, recent corroborating studies have found this may be driven through novel oxidative pathways.

Consistent in all such above approaches are the need to create highly controlled, spatiotemporal RF magnetic fields. In application, these fields should be distributed across living systems as required for varying applications in local drug release, cell stimulation, device powering or more. However, modern biological RF power systems are often simplistic and incapable of such complexity. Mobile RF systems deliver energy from a single coil to a single position (i.e., a Qi charger or reverse charging capabilities on a phone), while high-power systems are typically tethered to directly to the immediate rigid coils/electromagnetics of bulky power amplifiers. New, lightweight, mobile/flexible techniques to deliver RF magnetic power could unlock the potential of many emerging technologies, enable on-demand, deep tissue modulation. These could enable magnetic-driven dosing on demand that track alongside the user. Low-burden, low-cost techniques could yield new, accessible cancer or magnetic cranial imaging/stimulation treatments. Non-invasive deep brain stimulation in in-vivo models (such as mice) could be improve through lightweight interfaces that could partially untether animals from power supplies. Finally, such approaches could create networks of light, implantable devices all freed from bulky batteries.

As further described below, magnetic metamaterials may facilitate the delivery of RF magnetic power. In some embodiments, magnetic metamaterials may include arrays of inductors that may support the propagation of magnetoinductive (MI) waves along its pathway (forming a waveguide). Such MI waves may exhibit comparatively low loss in attenuating media and may be utilized as an approach to spatially transport waves across soil, salt water/ocean, during MRI, and along the body. Despite its potential, many such investigations have primarily been theoretical or in small testbeds—this is because in practice inductors orient perpendicularly and are difficult to directly implant into environments. In addition, the functional distance of RF magnetic power transported by these waves may be relatively low—this is because the entire waveguide is homogeneous, active, and radiates in the nearfield.

Although specific applications of RF magnetic field in biomedical technologies are discussed above with respect to FIG. 1, any of a variety of applications of RF magnetic fields as appropriate to the requirements of a specific application can be utilized in accordance with embodiments of the invention. Clothing integration and multi-sensor studies in accordance with embodiments of the invention is discussed further below. Magnetic metamaterial networks in accordance with embodiments of the invention are further described below.

### Magnetic Metamaterial Networks

The present embodiments may include next-generation magnetic metamaterial networks that enable powerful, new domains of electromagnetic control in living systems, such as, but not limited to, the body. The present embodiments may be accomplished through combined innovations in electromagnetic design and materials integration, as further described below. In many embodiments, structures may be heterogeneous, low-loss, flexible, and include optional active cooling elements. Such structures may allow the coordinated delivery of significant RF power at varying frequencies to key localities, while maintaining high transmissivity over long distances. Various potential applications include, but are not limited to, powering of complex networks of wearable or implantable sensors (these may be free from costly batteries), localized frequency-selective delivery or activation of magnetic particles (e.g., for drug release or cell/neuronal control), and non-invasive deep tissue (e.g., brain) stimulation or imaging partially-untethered from bulky power supplies.

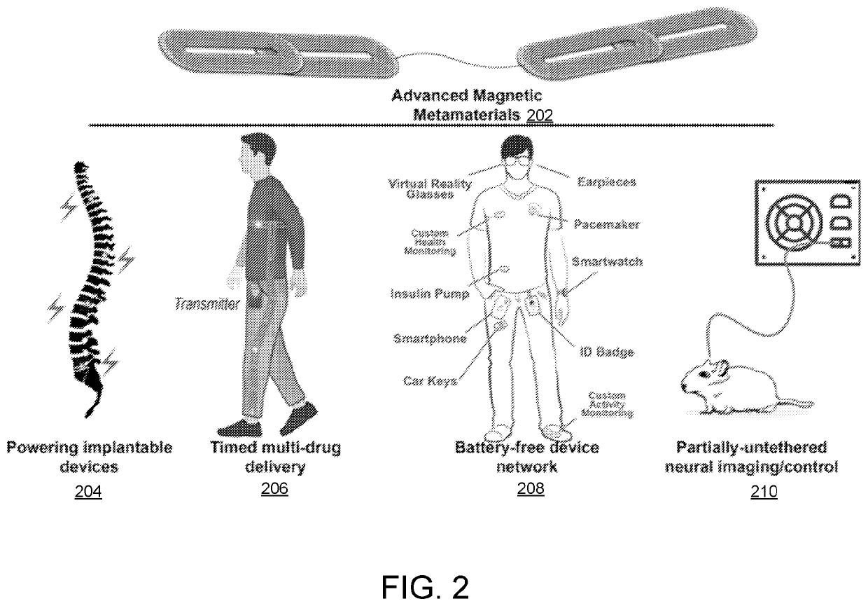

A diagram illustrating implementations of magnetic metamaterials in functional biomedical technologies in accordance with an embodiment of the invention is shown in FIG. 2. Advanced heterogeneous magnetic metamaterials 202 enable new domains of electromagnetic control in living systems. In many embodiments, magnetic metamaterials 202 may include one or more planar inductive elements that exhibit programmability in design and architecture, as further described below. As described herein, magnetic metamaterial networks may be constructed on demand and optimized for specific applications. For example, magnetic metamaterial networks 202 may be utilized for various applications, including, but not limited to, powering implantable devices 204, timed multi-drug delivery 206, battery-free networks 208, partially-untethered neural imaging/control 210, etc. In various embodiments, magnetic metamaterial networks may modulate the transmission of MI waves. In some embodiments, magnetic metamaterial networks may include controllable regions of near-field radiative and low-loss non-radiative sections enabling signal to pass long distances without losing amplitude while maximizing effect. In some embodiments, magnetic metamaterial networks may include magnetic skyrmions allowing magnetic metamaterials to transmit multiple, engineered frequencies (examples are further described below in the “Preliminary Data Considerations” section, passing multiple NFC and power transmission frequencies). In some embodiments, magnetic metamaterial networks may include mixed inductive components that may modulate the local penetration depth or surface uniformity of RF signals. As further described below, magnetic metamaterial networks may enable the unique routing of RF magnetic field for emerging networks, such as, but not limited to, in biomonitoring or bioactuation.

In many embodiments, advanced mechanical/material substructure may enable high RF magnetic power delivery alongside complex living structures. To deliver power alongside living systems metamaterials should either be flexible or pre-shapeable, yet additionally must maintain low-loss, thermal energy dissipative geometries—this enables large fields for those applications that need it. The present embodiments investigate several strategies to enhance signal delivery through conductance. In RF transmission, loss may become limited by the skin-depth of the conductor. Advanced flexible structures may be composed of multi-layers of copper/silicone to maximize conductance while maintaining flexibility. Optional embedded silicone fluidic channels may allow ice-cold water to further cool the structure and allow high-power transmission. For ultra-high power needs (e.g., for techniques in deep brain drug delivery or stimulation), the present embodiments may include thermally-cooled Litz wires. These may combine litz/bundle wires with interspersed fluidic tubing to cool the wire allowing optimal transmission of power while retaining high surface areas for efficient cooling.

Although specific manifestations of magnetic metamaterials in functional biomedical technologies are discussed above with respect to FIG. 2, any of a variety of manifestations of magnetic metamaterials as appropriate to the requirements of a specific application can be utilized in accordance with embodiments of the invention. Preliminary data considerations in accordance with embodiments of the invention are discussed further below.

### Preliminary Data Considerations

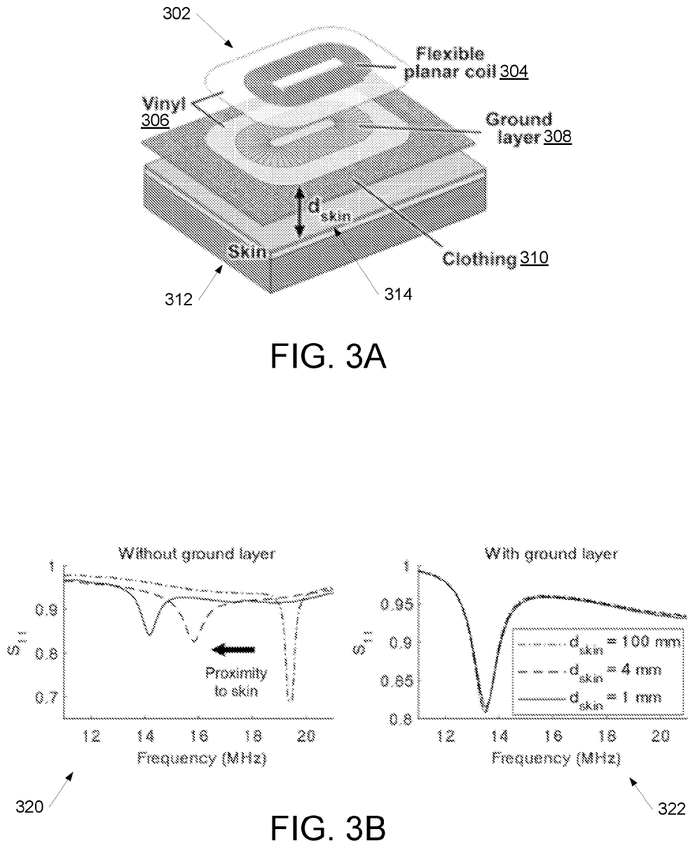

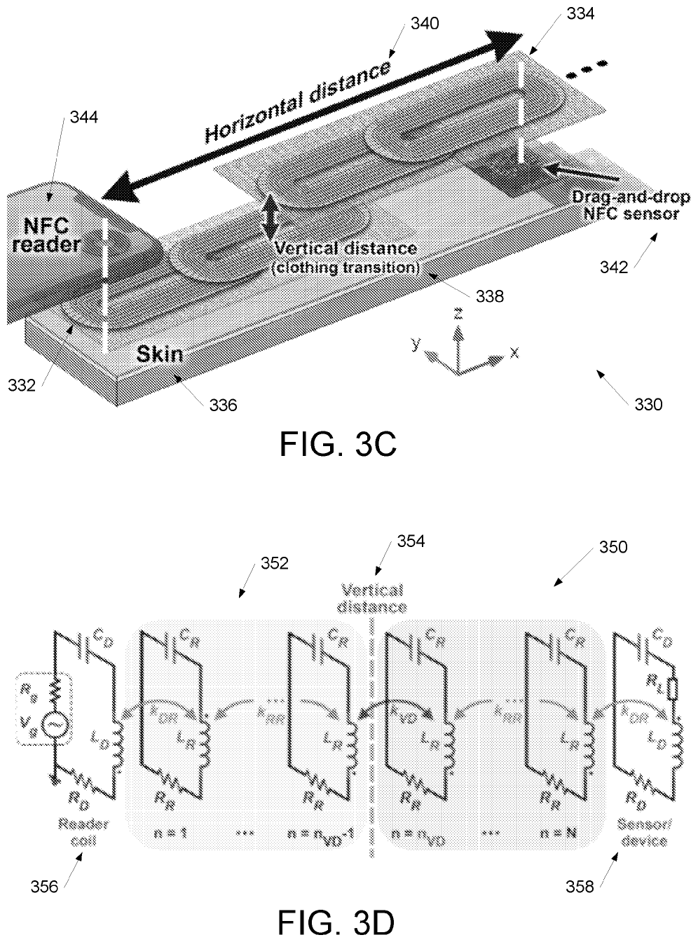

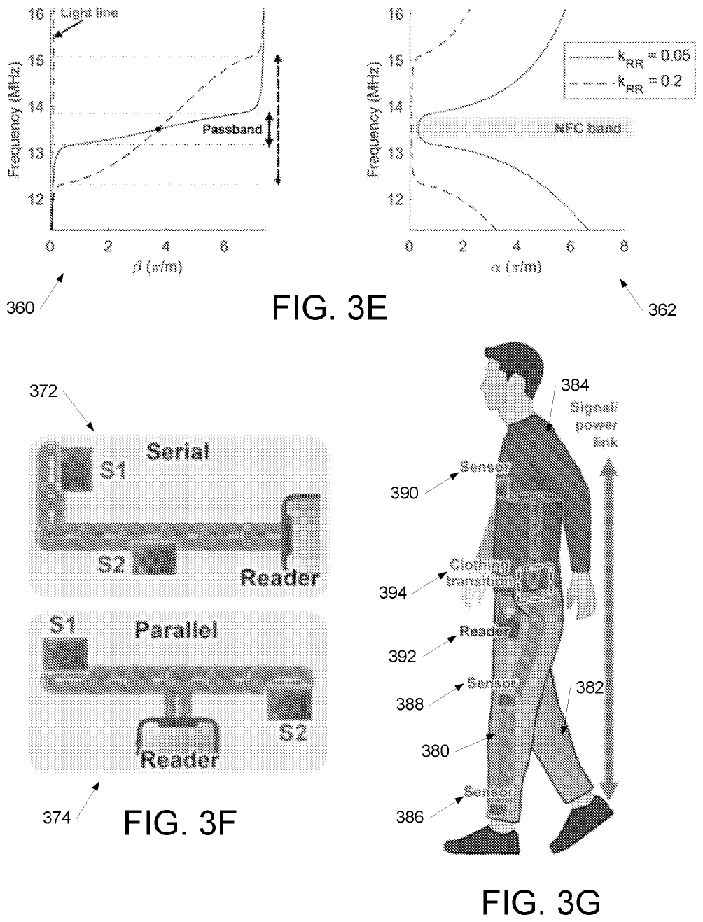

Preliminary data considerations include, but is not limited to, functional magnetic metamaterial networks for controlling biosignal transfer, alongside validation of several advanced proposed magnetic structures. Diagrams illustrating magnetic metamaterial integrated textiles enabling on-body transfer of nearfield signal power in accordance with an embodiment of the invention are shown in FIGS. 3a-g. A schematic diagram illustrating a planar magnet resonator in accordance with an embodiment of the invention is shown in FIG. 3a. The planar magnet resonator 302 may include a flexible planar coil 304 and a ground layer 308. In some embodiments, the flexible planar coil 304 and/or the ground layer 308 may be on vinyl 306. The planar magnet resonator 302 may be placed on top of clothing 310 that may be on a person's skin 312. The distance 314 between the skin and the resonator 302 is shown. Graphs 350, 360 illustrating a ground layer minimizing spectral uncertainty due to a human body's parasitic effect in accordance with an embodiment of the invention is shown in FIG. 3b. Specifically, graph 320 is without ground layer and graph 322 is with a ground layer. In many embodiments, this may compensate for the power dissipation generated from the flow of image currents on the ground layer. Therefore, the slotted ground layer may intervene in between the loop and skin, eliminate the unpredicted spectral shift of the resonator, and help to miniaturize the loop while not significantly affecting the resonator's quality factor compared to when the loop is directly put on the skin.

The magneto-inductive waves can propagate through more convoluted pathways involving arrays of magnetically coupled resonators. A schematic diagram illustrating NFC sensors that may be dragged and dropped across the magnetically coupled resonators with a horizontal distance (in x-direction) and a vertical distance in other directions in accordance with an embodiment of the invention is shown in FIG. 3c. Diagram 330 includes magnetically coupled resonators 332, 334, on skin 336, having a horizontal distance 340 and a vertical distance 338. The NFC sensor(s) 342 may be dragged and dropped across the magnetically coupled resonators 332, 334. In various embodiments, this magnetic connection allows for more flexibility in terms of the resonators' 332, 334 relative placement and introduces a horizontal distance 340 within a network between the NFC reader 344 and sensor nodes (in x-direction), in addition to the vertical distances 338 (VD) between two neighbor nodes (resonator/device) on different pieces of clothing (or z axis). Such networks show propagation behavior along the coils (x direction) and typical near field properties in other directions. Thus, the nodes (including reader and multiple sensors) in the close vicinity of the coil network would be magnetically connected. The network's equivalent circuit 350 comprised of N coupled coils 352 plus one reader 356 and sensor 358 with a vertical distance 354 in between is shown in FIG. 3d.

The present embodiments may assume that the current flowing in the n^{th} resonator has a sinusoidal time-dependency with an angular frequency of w. Here, the resonator-coils each with an impedance of

$${Z}_{R} = {R}_{R} + j ω {L}_{R} + \frac{1}{j ω {C}_{R}} ,$$

may be inductively coupled to their closest neighbor resonator with the mutual coupling of M_{RR}=k_{RR}L_{R} where M and k represent the mutual inductance and coupling factor frequency respectively (index RR shows inter-resonator relations). In many embodiments, the resonators form a linear array with an equal distancing of d_{c} between two neighbor coils. For simplicity, it may be assumed the vertical distance is ignorable (k_{VD}=k_{RR}). Further, the current running on the n^{th} resonator (ranging from 1 to N) in an array can be represented by:

$${I}_{n} = {I}_{1} {e}^{j {ϕ}_{1}} {e}^{- j γ ( n - 1 ) {d}_{c}} \qquad ( 1 )$$

where γ is the travelling wave's propagation constant, I₁ and ϕ₁ are the first loop's current magnitude and phase depending on the excitation (boundary conditions imposed by reader's V_{g}). The Kirchhoff's voltage law for the n^{th} coil follows:

$${Z}_{R} {I}_{n} + j ω {M}_{RR} ( {I}_{n - 1} + {I}_{n + 1} ) = 0 \qquad ( 2 )$$

which leads to the dispersion equation:

$$γ = \frac{1}{{d}_{c}} \times {cos}^{- 1} ( \frac{- {Z}_{R}}{2 j ω {M}_{RR}} ) \qquad ( 3 )$$

This structure may support forward and backward traveling waves and thus form a standing wave along the resonators array. To match the standing wave's spatial harmonics with the array's geometry, we define γₘ=4γ/m where m (>1) indicates the number of coils between two spatially equal-phase planes along the standing wave. This enables analyzing the propagation characteristics per unit of resonator (instead of length) and would be ultimately helpful to identify the resonator number on which the standing wave's peak places. The per-resonator expression of the spatial harmonics facilitates the network's design for the end user to plan the number of resonators on each piece of clothing and thus optimize the BAN. Here, γₘ=β−jα is the harmonic propagation constant (β as the phase and α as attenuation constants) and is calculated for our typical resonator properties and shown in FIG. 3e. Dispersion diagrams 360, 362 for an array of resonators for various magnetic coupling coefficients in accordance with an embodiment of the invention is illustrated in FIG. 3e. The lower and higher cutoff frequencies (MI wave passband) are marked by dotted lines and specify the bandwidth. The light line (with a large slope of the light velocity in free space) is shown by the dashed line.

Diagrams illustrating a reader and multiple sensors utilized in an in-line serial 372 and a T-shaped parallel 374 array of resonators to form various signal paths around a body in accordance with an embodiment of the invention are shown in FIG. 3f. When integrated into clothing, it may allow for the BAN's complex signal paths to span across multiple layers of disconnected clothing (e.g. from pants to shirts), distinguishing itself from other textile-BANs that rely on a wire- or conductive thread-based connection. A diagram illustrating a metamaterial network 380 streamlined into separate clothing pieces in accordance with an embodiment of the invention is shown in FIG. 3g. The metamaterial network 380 may be easily streamlined into separate clothing pieces (e.g., pants 382 and shirt 384 having a clothing transition 394), enabling high flexibility necessary for daily routines and significant horizontal range extension. The metamaterial network 380 may include various components such as, but not limited to, sensor(s) 386, 388, 390 and reader(s) 392. The nodes may be placed anywhere close to (within a few centimeters of) any point of the network.

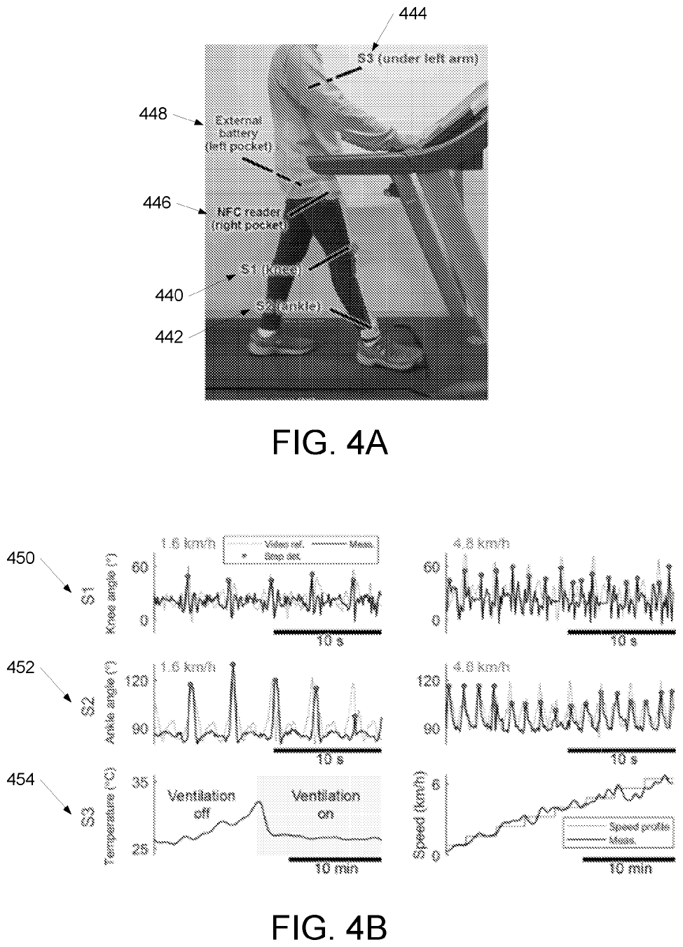

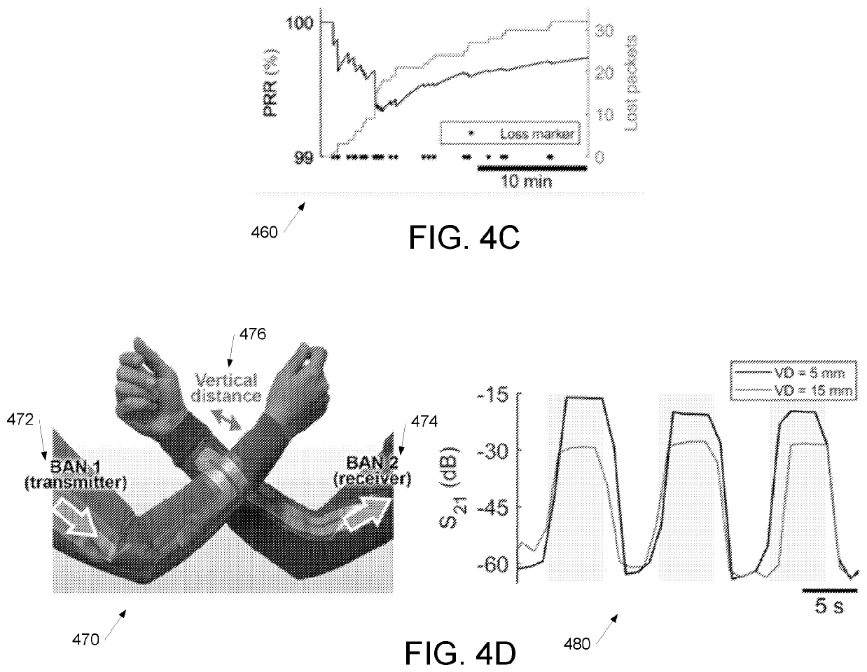

Diagrams illustrating in vivo multi-transponder and multi-BAN communication by textile-integrated waveguides in accordance with an embodiment of the invention are shown in FIGS. 4a-d. FIGS. 4a-c illustrate network stability and characteristics that were validated over 30 minutes of run exercise. Specifically, a high speed, long-term indoor walk/running activity measurement in accordance with an embodiment of the invention is shown in FIG. 4a. In many embodiments, test may be performed under a gradually increasing velocity profile for 25 min and BAN may be integrated into clothing with colored vinyl. In various embodiments, the BAN may include sensors 440, 442, 444, and NFC reader 446. In some embodiments, the BAN may also include an external battery 448. Diagrams 450, 452, 454, monitoring of sensors 440, 442, 444, respectively, during indoor running under various velocity profiles with a sampling rate of 10 Hz/sensor in accordance with an embodiment of the invention is shown in FIG. 4b. In various embodiments, steps may be detected and marked by circular markers. Diagram 460 illustrating long-term packet loss monitoring during indoor running in accordance with an embodiment of the invention is shown in FIG. 4c.

FIG. 4d illustrates that the network enables multibody transfer of power/signal. The surface propagation characteristics of the magneto-inductive structures enable seamless body-to-body communication with no need for terminals. The link readily establishes by putting any point of the two contributing BANs close enough (similar to the vertical distance between different pieces of clothing as shown in FIG. 4d). A diagram 470 illustrating body-to-body communication enabled by NFC's plug-and-play characteristics and its measured transmission during dragging the hands close and far over time (comprising an action of a “digital high-five”) for various VD values in accordance with an embodiment of the invention is shown in FIG. 4d. The body-to-body communication may include a first BAN (transmitter) 472 and a second BAN (receiver) 474 having a vertical distance 476. The external near-field communication may similarly be generalized to nearby local networks integrated into, for example, driver seats or gateways for monitoring and authentication purposes. The graph 480 illustrates data for VD=5 mm and 15 mm.

Data considerations include on-demand, textile-integrated magnetic metamaterials for multi-body area networks. Demonstrations show an iteration of the present embodiments magnetic metamaterials may be implemented on textile to enable battery-free, body area networks. Such power/signal facilitation illustrate a viable application of the present embodiments. For example, MI waves may propagate through an array of magnetically coupled resonant structures that possess equivalent spectral characteristics. The resonators may be designed in variant forms depending on the network's desired characteristics. Here, a desired body area network imposes a narrow set of constraints on the metamaterial array, such as high degrees of flexibility, being easy to extend, and possessing a microelectronic-free design. Multiturn flexible planar coils made of metal (e.g., aluminum and/or copper) foils may be used as resonators to be integrated into plastic film or textile. To remove the effect of the tissue on the resonant frequency of the coil, the present embodiments can either create a ground shield layer or utilize a relatively large lumped capacitance to stabilize the resonant frequency. The performance of such structures remain stable during various mechanical distresses such as flexing and bending. A facile and versatile technique of integrating metamaterial railways was proposed. The coil trace and slotted ground layer were cut out of copper aluminum foils. Layers were stacked, then placed on the clothing, and finally fixed by heat pressing.

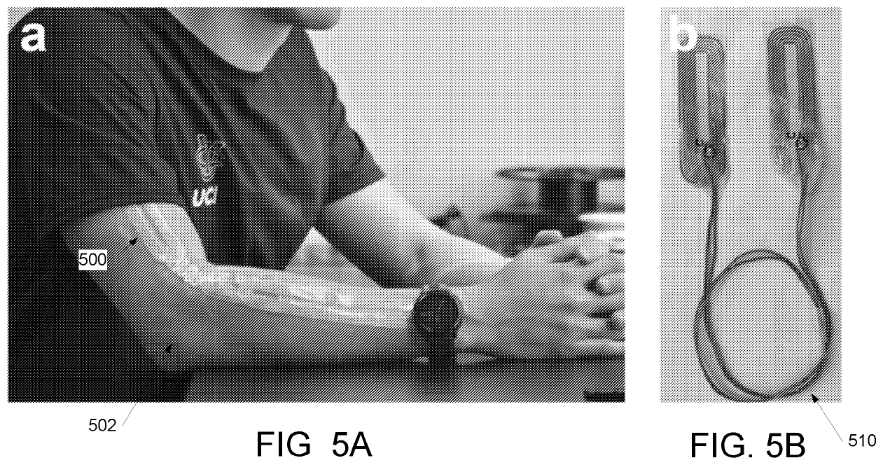

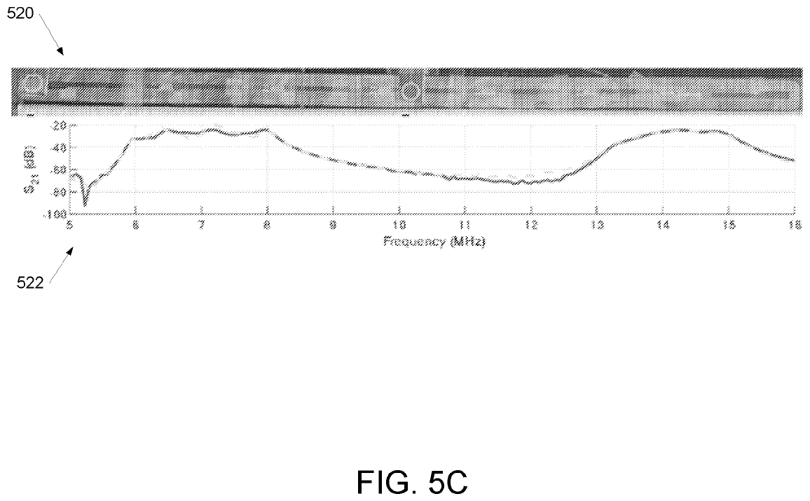

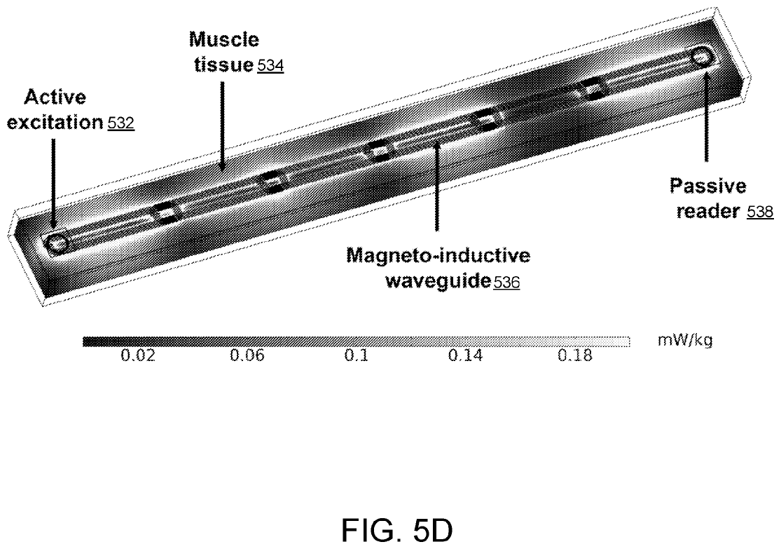

The present embodiments may include similar strategies to create skin-borne networks, which may be scaffolded on tegaderm. FIGS. 5a-d are diagrams illustrating advanced magnetic metamaterials in accordance with an embodiment of the invention. FIG. 5a illustrates an epidermal magnetic metamaterial 500 placed directly on the skin 502 of a user. FIG. 5b shows a “jumper” metamaterial element 510. Further, FIG. 5c illustrates an engineered multi-passband metamaterial element 520 and a graph 522 illustrating its spectral characteristics. In addition, FIG. 5d illustrates a multi-passband metamaterial element 530 showing active excitation 532, muscle tissue 534, magneto-inductive waveguide 536, and a passive reader 538. FIG. 5d also shows that SAR at 1 W at transmitter exhibits 2 orders of magnitude lower SAR than allowable limits.

The versatility of this network was evaluated by various readers and sensors (including off-the-shelf and an optimized board design) shown in FIG. 4f, that can be placed close (within the vertical distance of 3 cm) to any point of the resonator chain. This initial sensor board is based on a commercially available NFC transponder chip integrating an analog to digital unit that enables us connect a wide range of analog sensors, such as, but not limited to, strain and temperature sensors. MI structures enable seamless body-to-body communication with no need for terminals. The link readily establishes by putting any point of the network close enough, because of the same behavior as the vertical distance between different pieces of clothing. The external nearfield communication may similarly be generalized to nearby local networks integrated into, for example, driver seat or gateways for monitoring and authentication purposes. The network's wireless efficiency was examined by measuring the NFC packet reception ratio (PRR).

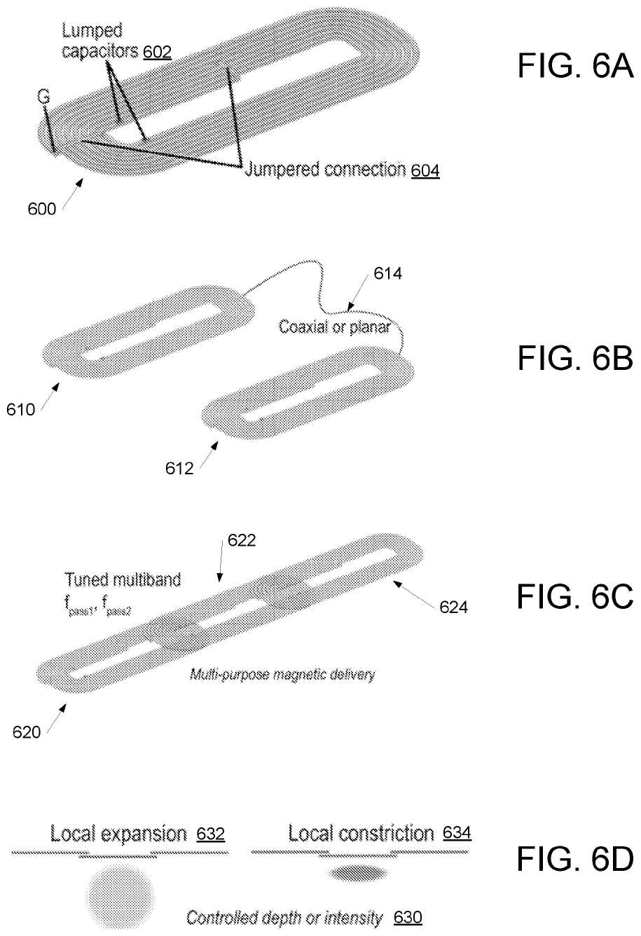

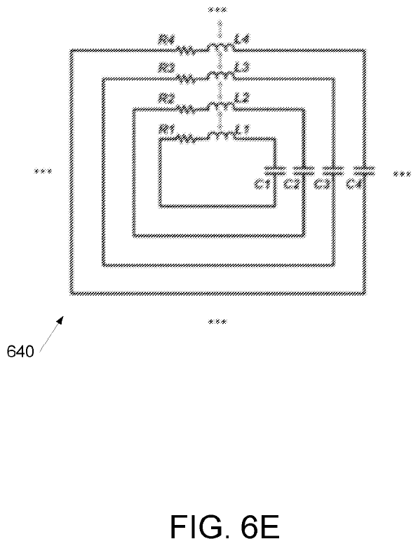

Sensor networks were characterized during various strenuous daily activities, and linking multi-bodies. Diagrams illustrating magnetoinductive elements for MI wave manipulation in accordance with an embodiment of the invention are shown in FIGS. 6a-e. As illustrated in FIG. 6a, a magnetoinductive element may be configured as a flexible element 600 that may include lumped capacitors 602 and jumpered connections 604. In many embodiments, flexible elements may be readily tuned for specific frequency or loss. Further, as illustrated in FIG. 6b, a jumper 614 may enable long distance/low-loss connectivity between flexible elements (e.g., flexible elements 610, 612). Moreover, as illustrated in FIG. 6c, multi-band may enable multi-frequency transmission using flexible elements (e.g., flexible elements 620, 622, 624). Furthermore, FIG. 6d illustrates a controlled depth/field focusing 630 having local expansion 632 and/or local constriction 634. In addition, FIG. 6e shows a uniformed area stimulation 640.

The present embodiments may include advanced magnetic metamaterials for multi-parametric control of spatiotemporal RF magnetic fields. Validation for metamaterials exhibiting advanced control of magnetoinductive waves have been performed. Two examples are highlighted here, that demonstrate the present embodiments control of spatial distribution and wave frequencies. First, the present embodiments demonstrate controlled regions of nearfield radiative and non-radiative by interceding an inductive element that may be referred to as a “jumper.” Here, two small coil elements may be connected over long range by a low-loss connection (this can be through simple planar wires or coaxial cables-very low loss). When interceded into the network as an element, wave propagation is still supported, however a null point appears as the wave effectively disappears and reappears across the arbitrarily long points of the jumper wire. In practice, this element may allow RF fields to spatially localize to desired points at arbitrarily long distances away. The present embodiments also include a second structure for the support of engineered frequency bands. One of skill should appreciate resonant circuits may support multiple modes. However, these modes typically appear at set frequencies as determined by the resonator geometry and cannot be controlled. The present embodiments have developed multiple strategies that enable the propagation of multiple engineered frequencies, one of which is highlighted here for exemplary purposes. By interceding a tuning capacitor within the element, the present embodiments can engineer the passband of the metamaterial to propagation specific designer frequencies (such as, but not limited to, 300 kHz/NFC, 6.7 MHz/NFC). This will allow enhanced levels of control in terms of magnetic power, stimulation, or information transfer. Note, metamaterials of the present embodiments exhibit low SAR due to minimal electric field propagation.

Although specific preliminary data considerations are discussed above with respect to FIGS. 3a-6e, any of a variety of data considerations as appropriate to the requirements of a specific application can be utilized in accordance with embodiments of the invention. Considerations and approaches for magnetic metamaterial networks in accordance with embodiments of the invention are discussed further below.

### Considerations and Approaches for Magnetic Metamaterial Networks

The present embodiments may be utilized for next-generation applications in magnetic imaging, drug delivery, implantable power, deep brain stimulation, and more. The present embodiments may include design, model, build, and validate such structures that can deliver spatiotemporally engineered, bioactuating RF magnetic power alongside living systems.

In many embodiments, MI wave-modulators for spatio-temporal control of RF magnetic fields may be considered. Magnetoinductive waves traditionally propagate via a static, linear array of inductors that exhibit repetitive structural/spectral characteristics. The most common manifestation is as split ring resonators that orient in perpendicular or planar arrangements (usually on a printed circuit board (PCB)). Such geometries may be exploited in various commercializing technologies to enhance moderate-range power transfer. While useful, existing devices have fairly-limited application in biological technologies because structures cannot easily be directly implanted into or alongside living systems. The present embodiments introduce several EM innovations that introduce significantly more programmability/versatility to such components, thus enabling novel biologically-interacting structures. For example, such networks may include: 1) metamaterials that can be inhomogeneous, 2) inductive elements that are both readily and uniquely tuned—in addition their orientation can be adapted to synthesize networks on-demand, and 3) control components integrated within the metamaterials.

The present embodiments include the design and optimization of inductive elements tuned to modulate MI waves in unique ways within networks. Here, the fact that MI waves can be transported so long as the frequency response of individual elements exhibit similar behavior may be leveraged. This frees the present embodiments from the static network that typically composes a MI waveguide. Inductive structures may be exchanged within the network to perform specific tasks or to optimize the performance of the network.

The present embodiments may include the study and validation of jumper elements. This performs an important task as enabling MI waves to transfer long distances within a non-radiative waveguide. Two implementations may be investigated, a simple planar foil waveguide (that can be built directly alongside various designs), and a more low-loss iteration using a coaxial cable. The next elements may include those that enable the propagation of multiple frequency bands. Such structures may facilitate multiple lanes of control/power/signal. For example, one band could control hyperthermia or deliver power to devices (300 kHz), while another band passes signal or controls elements (NFC). Here two implementations may be considered, one where the higher order harmonics of a single coil to tuned to desired frequencies, and a second where nested coils are used, wherein each coil exhibits its own passband (technically these approaches can combine to form four or more frequency passbands). The next elements may be controlled depth/focusing. Here, because coils are planar and can be readily designed on-demand, the width can be modulated to either shrink or expand to fit potential application requirements. Due to the size variation, such elements may need specific tuning due to varying inductance. A variety of potential structural designs may be tested to optimize the coupling coefficient. Finally, for uniformed area stimulation the present embodiment may include examination of structures facilitating even stimulation over large areas (this would be ideal for drug release for example). Such elements would possess higher uniformity in comparison to either an individual coil or a metamaterial. Here, the present embodiment may include a spoof plasmon architecture, wherein individual single turn elements couple to its nearest neighbor.

The behavior of individual elements (of varying design) and performance within a network may be assessed using a variety of strategies. First, the present embodiments may include an advanced analytical model that can handle the unique parameters of various designs. For example, a matrix model may be utilized that can uniquely handle various metamaterials of the present embodiments. Each design may have its own accompanying matrix, and unique coupling parameters created by individual interlocking elements can be handled within matrix parameters. Various aspects such as, but not limited to, the propagating magnetic field and power can be extracted. Such a model can give a good first order approximation of metamaterial performance. Advanced behavior of the present magnetic metamaterial networks can be modelled using finite-element analysis. Such FEM may be utilized to assess the Figure of Merit (FOM) of various metamaterials in delivering required magnetic field or power to localities. The maximum magnetic field and power may be simulated and plotted against a number of conditions of the network, including the elements, radiating vs non-radiating region areas, depth/position of measurement, and more. This may aide in understanding fundamental limits, wherein the more previous regions that excite in the nearfield may limit the generated field (power) later along in the network. Here, the present embodiments include understanding the required input power to facilitate the stimulation of large regions/depth with sufficient energy. This input power may place functional limitations, because compact, wearable power supplies presently have a limit of around 50 to 100 W. Higher power would typically require benchtop/large systems that would partially tether the user to a location. In addition, the experimental behavior of individual elements may be measured and validated against simulations.

In various embodiments, advanced MI networks and biomagnetic controls may be considered. Typically, RF magnetic power systems utilize a single coil connected to a power supply—this forms the transmitter. This transmitter has various manifestations. For example, cellphones are outfitted with NFC or Qi protocol transmitters that deliver up to 10 W, while much larger/advanced power supplies are used to deliver higher powers as required for technologies such as MRI. Advanced magnetic metamaterials can facilitate the transfer of signal from transmitters over complex environments. As described above, this has a variety of potential technological applications. However more advanced applications (that require delivery of very large power), this metamaterial should exhibit not only lower loss, but more advanced cooling systems to counteract heat generated by the currents induced in the conductor. The present embodiments may include such structures.

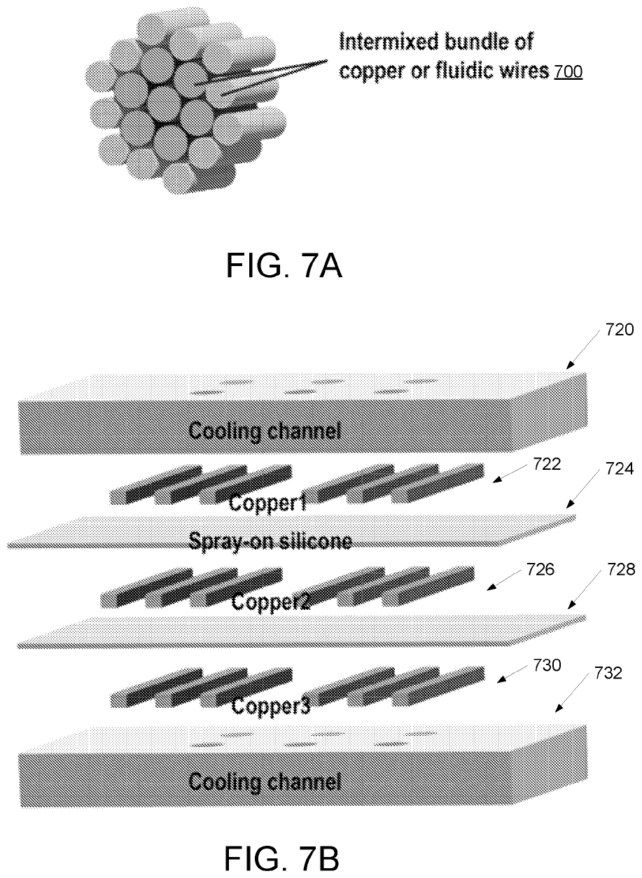

Currently, RF coils with embedded cooling utilize hollow copper windings, within which water is flowed to cool the structure. This structure is not an optimal one—this is because the resistance of the windings remains relatively high (which itself generates more heat), and cooling is not efficient. The present embodiments may include various implementations that may enable higher power MI waves to traverse on the present magnetic metamaterials. Diagrams illustrating construction of magnetic metamaterials with enhanced electronic properties and programmable thermal dissipation in accordance with an embodiment of the invention are shown in FIGS. 7a-b. As illustrated in FIG. 7a, a first implementation may remain flexible, utilizing multiple layers of foil (e.g., an intermixed bundle of copper or fluidic wires 700) to reduce coil resistance alongside flanking channels to facilitate active water cooling. As illustrated in FIG. 7b, a second implementation may include a modification of the bundle/Litz wire, which is commonly used to reduce the resistance of coils by utilizing conductors with diameter on the same order of the skin depth. In practice, Litz wire coils are not typically flexible, however they can be pre-shaped to mold over surfaces. By swapping a small percentage of individual wires with fluidic tubing, an efficient structure can be created that possess integrated cooling and low loss. Such a structure is additionally more versatile than hollow copper windings, because wire diameters can be swapped depending on application (higher frequencies require smaller diameter wires due to reduced skin depth. In reference to FIG. 7b, the second implementation may include a first cooling channel 720, a first copper layer 722, a first spray-on silicone layer 724, a second copper layer 726, a second spray-on silicone layer 728, a third copper layer 730, and a second cooling channel 732.

Various aspects of the present advanced conductors may be modulated and considered for their impact of coil electrical and mechanical properties. For example, for the first implementation shown in FIG. 7a, foil thickness, number, and spacing may be tested, while flanking silicone channels of varying thickness and reinforcement may be tested under mechanical flexing. For the second implementation shown in FIG. 7b, various sizes and ratios of wire and tubing may be tested. Designs may be excited with varying power of RF magnetic field, and heating during passive or active cooling may be considered. These considerations and approaches may give engineers an idea of limitations of various structures, and power at which cooling would be required to offset heat created within the conductors.

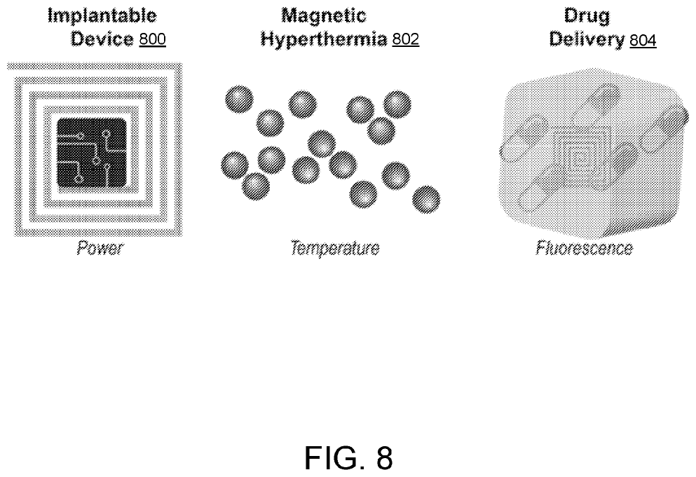

The present embodiments may include performing validations of magnetic metamaterials while performing major RF biomagnetic tasks. A diagram illustrating experimental validation of biomagnetic control in advanced magnetic metamaterials in accordance with an embodiment of the invention is shown in FIG. 8. In some embodiments, this may serve to benchmark the performance of the various generated devices in accordance with embodiments of the invention. First is implantable devices 800 including benchmarking for delivered RF power at various depth and positions along the network. This may be tested at major RF power bands/protocols, including NFC (13.56 MHz), AirFuel (6.5 MHz), and Qi (200 kHz). Second is magnetic hyperthermia 802. A variety of magnetic nanoparticles (Micromod, Ocean, all validated previously for use in hyperthermia) may be tested from vendors for their ability to induce spatially-localized heating through the present coil designs. Lastly, the present embodiments may include consideration of drug release 804 induced through hyperthermia. For example, Fluorescent-Drug (e.g., gentamicin) loaded hydrogels may be synthesized within which a small pickup coil may be floated. Hydrogels may be placed at varying distance and depth from the present metamaterials, and drug release may be measured and considered.

Although specific considerations and approaches for magnetic metamaterial networks are discussed above with respect to FIGS. 7-8, any of a variety of considerations and approaches for magnetic metamaterial networks as appropriate to the requirements of a specific application can be utilized in accordance with embodiments of the invention. Surface-spread magneto-inductive arrays in accordance with embodiments of the invention are discussed further below.

### Surface-Spread Magneto-Inductive Arrays

As described herein, the present embodiments may include various magneto-inductive elements including, but not limited to, hybrid jumpered wires, multiband skyrmions, and 2 dimensional arrays. In many embodiments, such magneto-inductive elements may be different wave modulating elements that may be engineered on-demand and integrated into various types of networks to create spatio-temporally engineered nearfield radiations wherever a contactless signal/power transfer is needed.

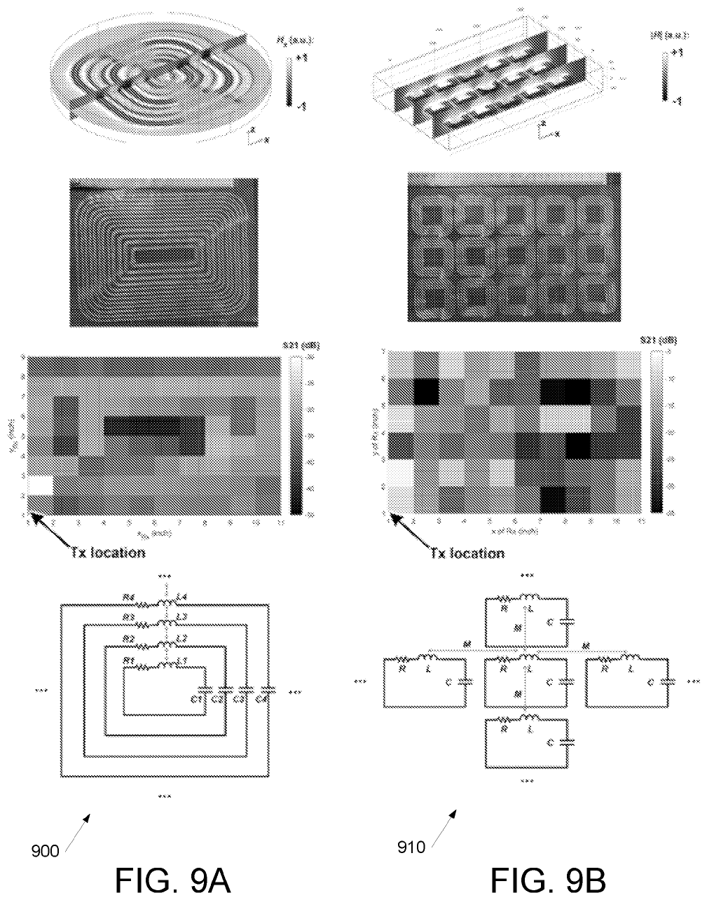

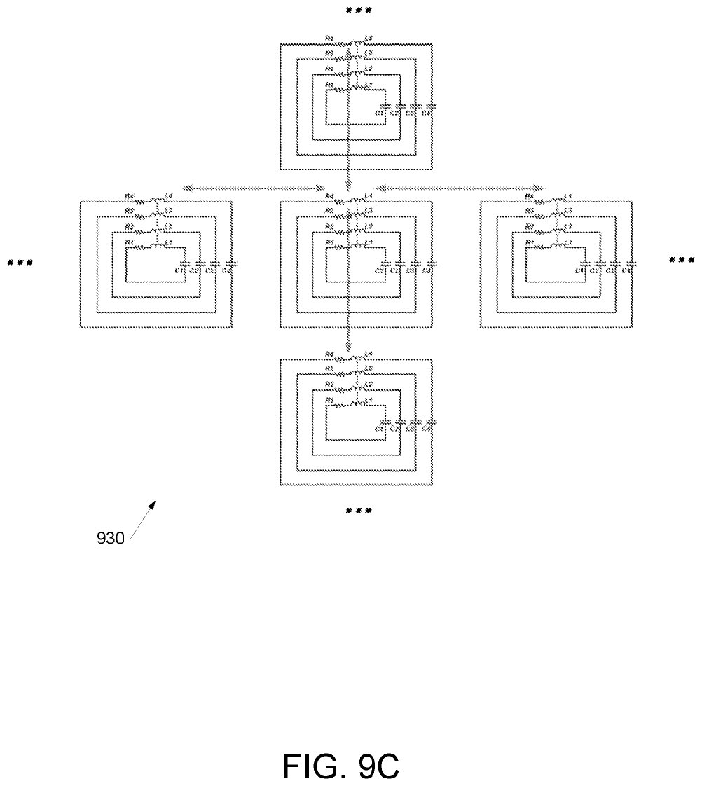

Diagrams illustrating surface-spread magneto-inductive arrays in accordance with an embodiment of the invention are shown in FIGS. 9a-c. In many embodiments, surface-spread magneto-inductive arrays can be formed into a nested colony 900 of small to large resonators with the same resonant frequency, as illustrated in FIG. 9a. Further, surface-spread magneto-inductive arrays can be formed into two-dimensional arrays 910 of identical resonators, as shown in FIG. 9b. In addition, surface-spread magneto-inductive arrays can be formed into a hybrid arrangement 920, as shown in FIG. 9c.

Although specific surface-spread magneto-inductive arrays are discussed above with respect to FIGS. 9a-c, any of a variety of surface-spread magneto-inductive arrays as appropriate to the requirements of a specific application can be utilized in accordance with embodiments of the invention. Epidermal magneto-inductive arrays and serpentine-integrated resonators in accordance with embodiments of the invention are discussed further below.

### Epidermal Magneto-Inductive Arrays and Serpentine-Integrated Resonators

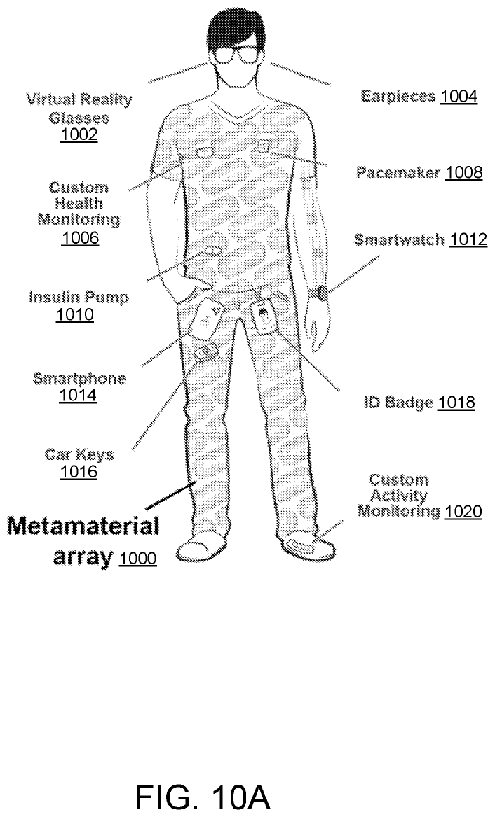

Diagrams illustrating wearable and epidermal variations of magneto-inductive metamaterials for human to peripherals for communication and powering applications in accordance with an embodiment of the invention are shown in FIGS. 10a-b. In FIG. 10a, potential peripherals (communicating through RFID and/or NFC protocols) a person may use daily is illustrated. For example, peripheral may include, but is not limited to, virtual reality glasses 1002, earpieces 1004, various custom health monitoring devices 1006, pacemaker, 1008, an insulin pump 1010, smartwatch 1012, smartphone 1014, car keys 1016, ID badge 1018, custom activity monitoring devices 1020, etc. In various embodiments, a potential surface-spread array of magneto-inductive metamaterials 1000 may be integrated into clothing. In reference to FIG. 10b, epidermal metamaterials 1030 may be attached to skin using a breathable waterproof sealing. Peripherals may include, but are not limited to, wearable or implantable electronics with nearfield magnetic coupling to a smartphone hub 1036.

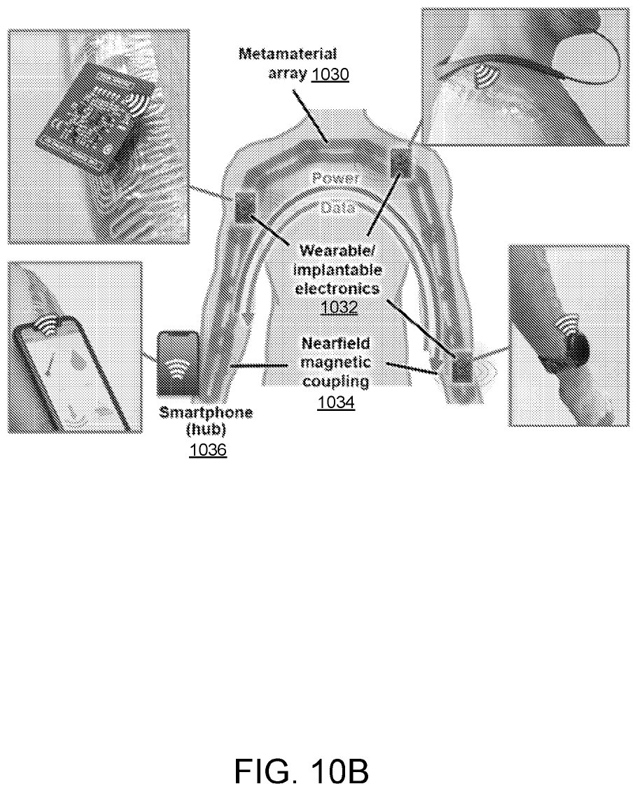

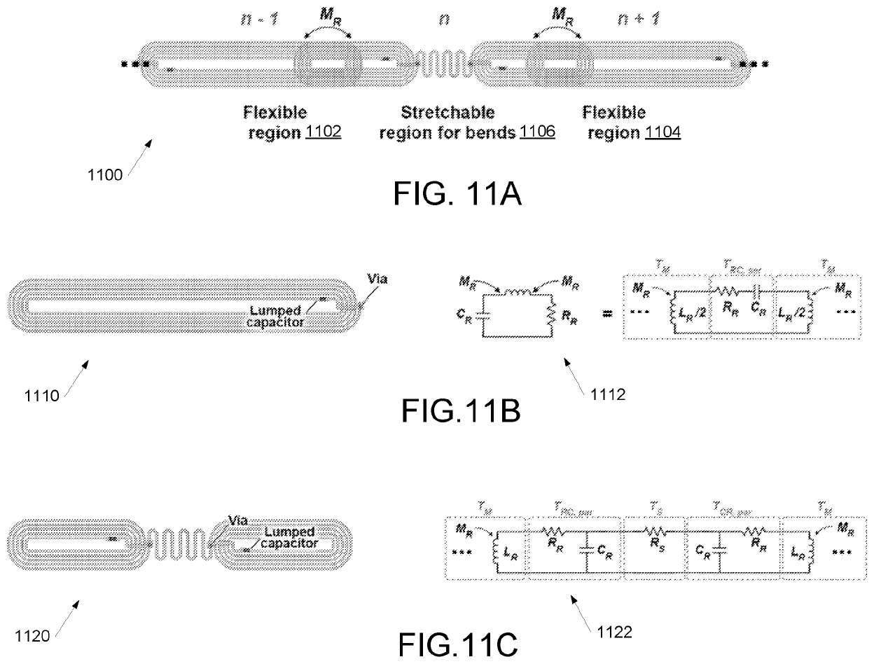

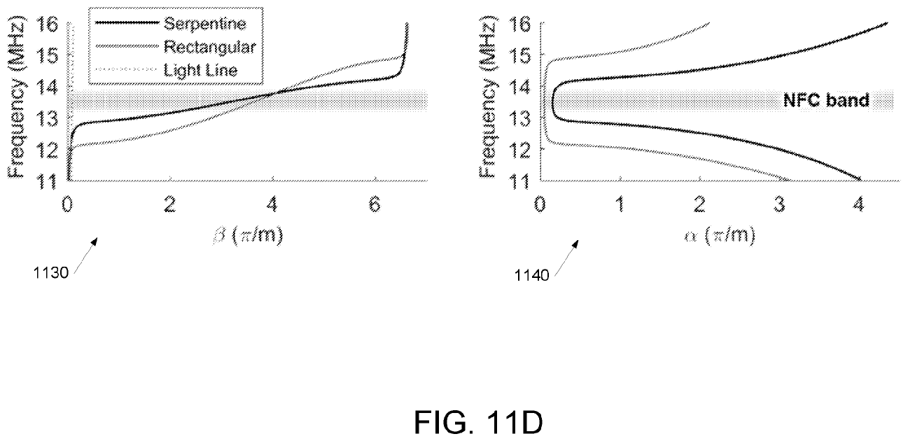

Assuming a length of d for neighbor resonator distances, the dispersion equation for the MI array can be calculated analytically. For a rectangular loop with an inductance, capacitance and resistance of L_{R}, C_{R}, R_{R}, the resonator's end-to-end transmission unit cell (ABCD matrix) for a resonator in a sufficiently long array is systematically derived by:

$${T}_{rect} = {T}_{M} \times {T}_{RC , ser} \qquad ( 4 )$$

in which T_{M} and T_{RC} are sub-network transmission matrixes (highlighted in FIGS. 11a-d) whose elements can be readily calculated in terms of the equivalent electrical components of the relevant sub-network. Similarly, for serpentine incorporated structure, the inductive and serpentine parts can be viewed as cascaded two-port sub-networks in form of:

$${T}_{serp} = {T}_{M} \times {T}_{RC , par} \times {T}_{S} \times {T}_{CR , par} \qquad ( 5 )$$

Components of the equivalent circuit model of both designs may be measured. In either case, assuming a sinusoidal waveform running on the resonant metamaterial elements results in:

$$[ {V}_{n + 1} {I}_{n + 1} ] = {T}_{equivalent two - port} \times [ {V}_{n} {I}_{n} ] = [ {V}_{n} {I}_{n} ] {e}^{- j γ d} \qquad ( 6 )$$

in which Vₙ and Iₙ respectively represent the voltage and current characteristics of the n^{th} resonator of a MI array network, and γ(=β−jα) is the traveling wave's propagation constant.

The analytical system eigenvalues (leading to the metamaterial's dispersion equation) can be solved by formatting (6) into a linear system of:

$$[ A - {e}^{- j γ d} BCD - {e}^{- j γ d} ] [ {V}_{n} {I}_{n} ] = [ 00 ] \qquad ( 7 )$$

in which A, B, C, and D are elements of the resonator's equivalent two-port network transmission matrix. The time-domain inductor's current at a desired n^{th} resonator is calculated by:

$${I}_{n} = {I}_{1} {e}^{j {ϕ}_{1}} {e}^{- j γ ( n - 1 ) d} \qquad ( 8 )$$

where I₁ and Φ₁ are the first resonator's current magnitude and phase depending on the excitation (boundary conditions), respectively. The comparison between the dispersion diagrams of metamaterial networks consisting of rectangular only and serpentine-incorporated only resonators shows a relatively larger bandwidth and lower loss associated with a rectangular design, because of its relatively larger mutual inductance and shorter length (thus ohmic loss). The serpentine-incorporated resonator, however, demonstrates significantly enhanced stretchability.

Diagrams illustrating a hybrid wired connection between neighbor resonators formed into a serpentine shape to enhance the stretchability and flexibility of the array particularly on the joints with frequent bends in accordance with an embodiment of the invention are shown in FIGS. 11a-d. A hybrid configuration 1100 is illustrated in FIG. 11a. The hybrid configuration 1100 may include flexible regions 1102, 1104, and a stretchable region 1106 for bends. A rectangular configuration 1110 and equivalent circuit representation 1112 are illustrated in FIG. 11b. Further, a serpentine-integrated array configuration 1120 and equivalent circuit representation 1122 are illustrated in FIG. 11c. A comparison of dispersion diagrams 1130, 1140 for a resonator distancing of 15 cm is shown in FIG. 11d.

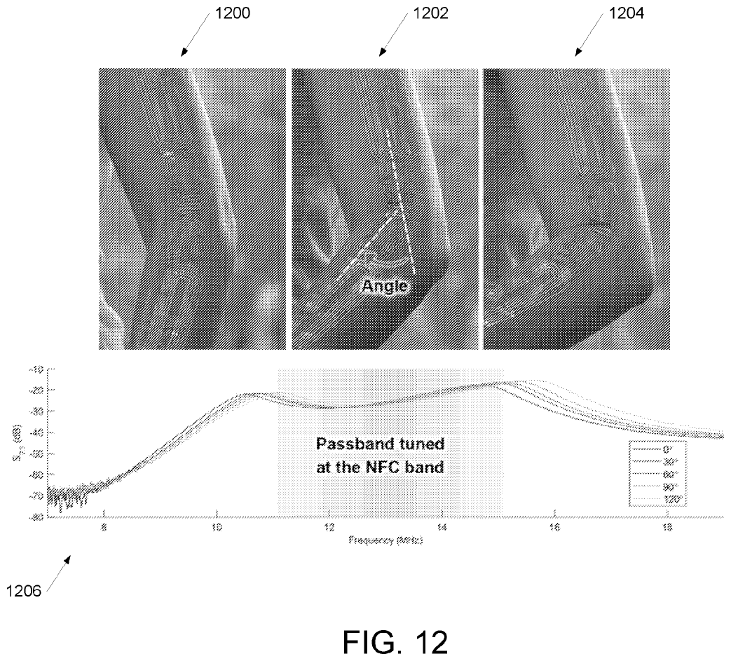

A diagram illustrating epidermal magneto-inductive metamaterials transferred onto human skin, tuned at NFC frequency in accordance with an embodiment of the invention is shown in FIG. 12. Transmission from shoulder to wrist (approximately 50 cm) under various elbow bending angles 1200, 1202, 1204 are illustrated. Further, a graph 1206 illustrating spectral characteristics of various elbow bending angles in accordance with an embodiment of the invention is shown in FIG. 12.

A diagram illustrating electrical reliability of cold-pressed capacitor joints for solder-free contact to copper traces in accordance with an embodiment of the invention is shown in FIG. 13. FIG. 13 illustrates transient monitoring of a single resonator's reflection coefficient with soldered 1300 and cold-pressed solder-free 1310 capacitor connections under continuous elbow bendings.

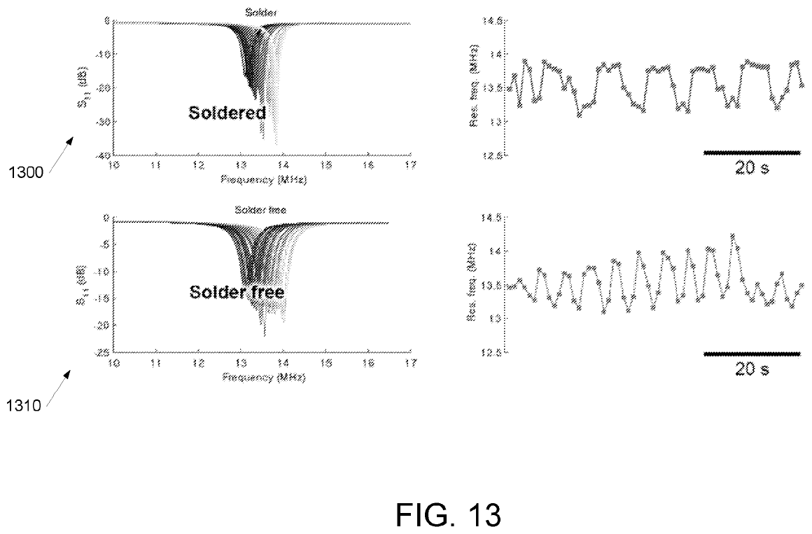

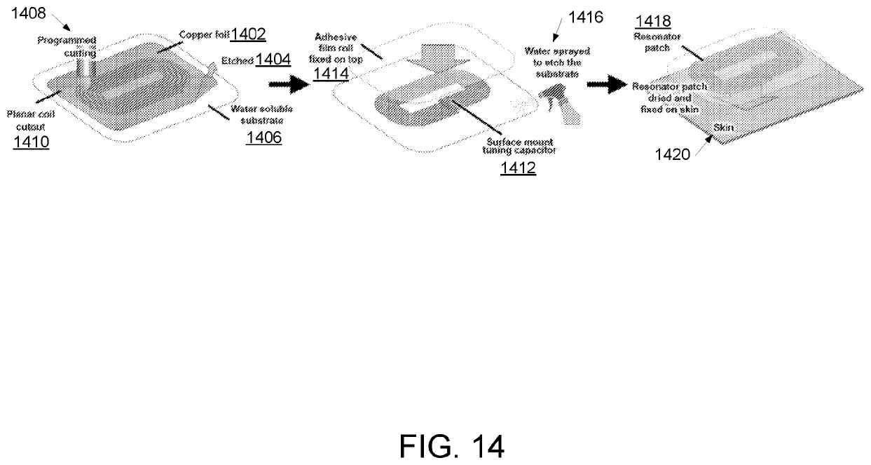

A diagram illustrating epidermal magneto-inductive metamaterial fabrication and skin transferring process in accordance with an embodiment of the invention is shown in FIG. 14. In reference to FIG. 14, a planar loop may be cut on a thin copper sheet. For example, a copper foil 1402 may be etched 1404 on a water soluble substrate 1406. In some embodiments, a programmed cutting process 1408 may be performed to produce a planar coil cutout 1410. Then, the tuning capacitor 1412 may be fixed by soldering or cold-pressing, and an adhesive film 1414 may be placed on top of the resonator, and the temporary substrate may be removed. In some embodiments, water may be sprayed 1416 to etch the substrate. Finally, the resonator patch 1418 is fixed on skin 1420.

Although epidermal magneto-inductive arrays and serpentine-integrated resonators are discussed above with respect to FIGS. 10a-14, any of a variety of epidermal magneto-inductive arrays and serpentine-integrated resonators as appropriate to the requirements of a specific application can be utilized in accordance with embodiments of the invention. Hybrid inter-connection of magneto-inductive arrays in accordance with embodiments of the invention are discussed further below.

### Hybrid (Wired and Wireless) Inter-Connection of Magneto-Inductive Arrays

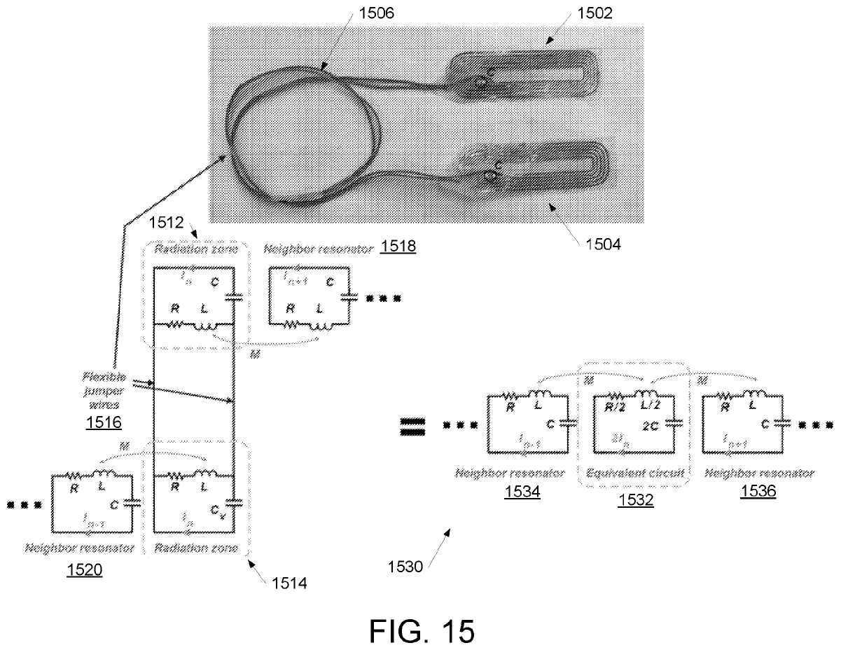

A diagram illustrating hybrid connection of two resonators with flexible jumper wires allows to create radiation-free blind spot and lower the magnetoinductive waveguide loss per unit of length in accordance with an embodiment of the invention is shown in FIG. 15. In many embodiments, a hybrid connection may include a first resonator 1502 and a second resonator 1504 with an inter-connection utilizing a jumper (e.g., flexible jumper wires 1504). In reference to FIG. 15, a circuit representation 1510 of the hybrid connection is provided. The circuit representation 1510 may include a first radiation zone 1512 (corresponding to the first resonator 1502) and a second radiation zone 1514 (corresponding to the second resonator 1504). The first and second radiation zones 1512, 1514 may be inter-connected using flexible jumper wires 1516. In some embodiments, the first radiation zone 1512 may be in proximity to a neighbor resonator 1518 and/or the second radiation zone 1514 may be in proximity to a neighbor resonator 1520. In further reference to FIG. 15, an equivalent circuit representation 1530 of the hybrid connection is provided. As illustrated, the equivalent circuit representation 1530 includes an equivalent circuit 1532 in proximity to the neighbor resonators 1534, 1536.

Although specific hybrid inter-connection of magneto-inductive arrays are discussed above with respect to FIG. 15, any of a variety of hybrid inter-connection of magneto-inductive arrays including wired and wireless approaches as appropriate to the requirements of a specific application can be utilized in accordance with embodiments of the invention. Multi-purpose, multi-band skyrmion arrays of magneto-inductive resonators in accordance with embodiments of the invention are discussed further below.

### Multi-Purpose, Multi-band Skyrmion Arrays of Magneto-Inductive Resonators

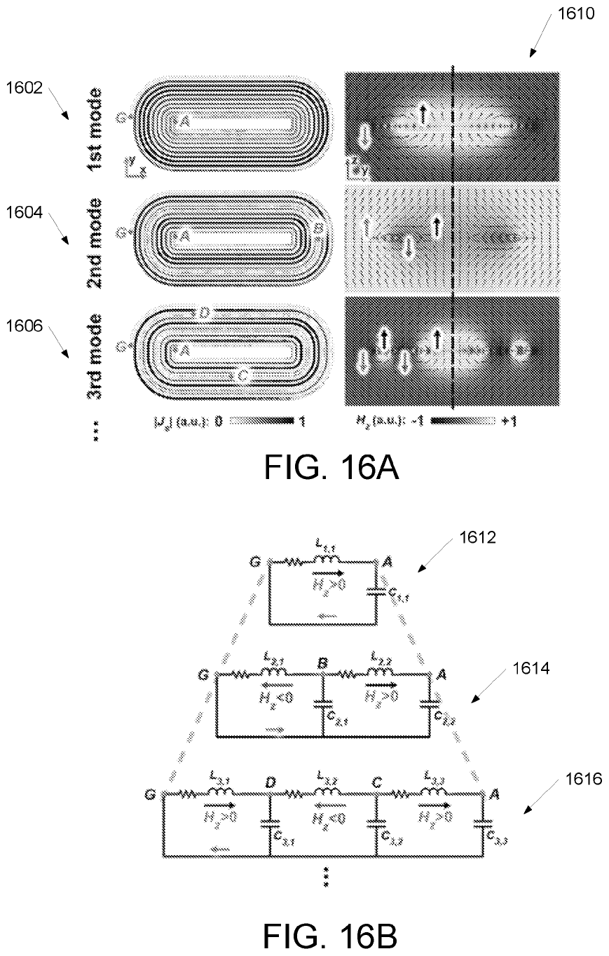

Diagrams illustrating demonstration of the fundamental and higher orders of resonance in the same multiturn loop, known as a “skyrmion” in accordance with an embodiment of the invention are shown in FIGS. 16a-b. The higher orders of resonance may include a first resonance mode 1602, a second resonance mode 1604, a third resonance mode 1606, etc. As illustrated diagram 1610 in FIG. 16a, in each resonance mode (e.g., first, second, and third resonance modes 1602, 1604, 1606), opposite direction of induced currents (each corresponding to a certain mode) disorients the generated magnetic fields locally. In FIG. 16b, the magnetic field corresponding to each mode is modeled using an equivalent circuit of higher order. For example, a first equivalent circuit 1612 corresponds to the magnetic field of the first resonance mode 1602, a second equivalent circuit 1614 corresponds to the magnetic field of the second resonance mode 1604, and a third equivalent circuit 1616 corresponds to the magnetic field of the third resonance mode 1606. Each resonant mode's frequency can be tuned using lumped capacitors integrated into various positions on the multiturn loop trace.

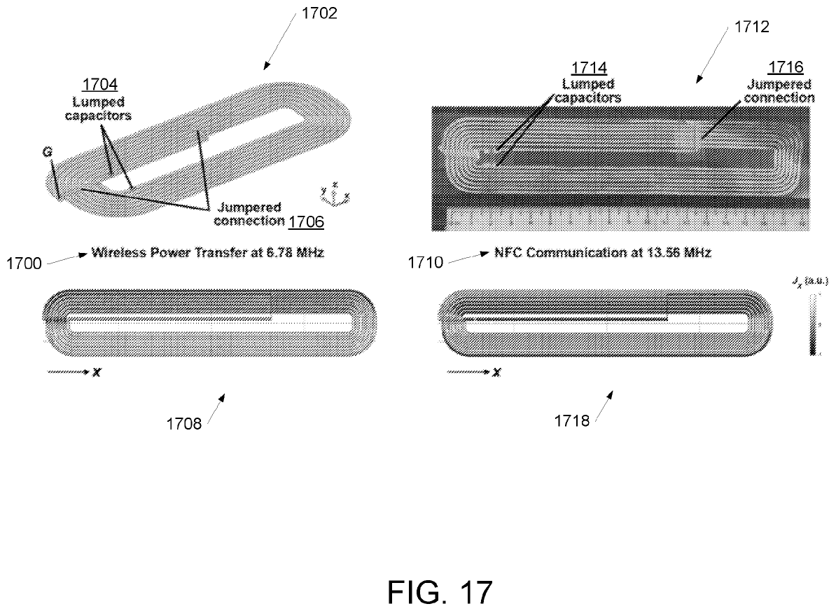

A diagram illustrating that one or more number of capacitor(s) may be placed at various positions on the multiturn loop to tune each resonance frequency in accordance with an embodiment of the invention is shown in FIG. 17. Wireless power transfer (WPT) at 6.78 MHz in accordance with an embodiment of the invention is shown in diagram 1700. The multiturn loop 1702 may include one or more lumped capacitors 1704 and jumpered connection 1706 placed at various positions to tune each resonance frequency. NFC communication at 13.56 MHz in accordance with an embodiment of the invention is shown in diagram 1710. The multiturn loop 1712 may include one or more lumped capacitors 1714 and jumpered connection 1716 placed at various positions to tune each resonance frequency. In some embodiments, minor traces may be needed to connect capacitors (e.g., capacitors 1704, 1714) to the right position. The surface current profile 1708 and 1710 of the first two modes (tuned into the WPT and NFC frequency bands), respectively, shows the currents corresponding to its mode.

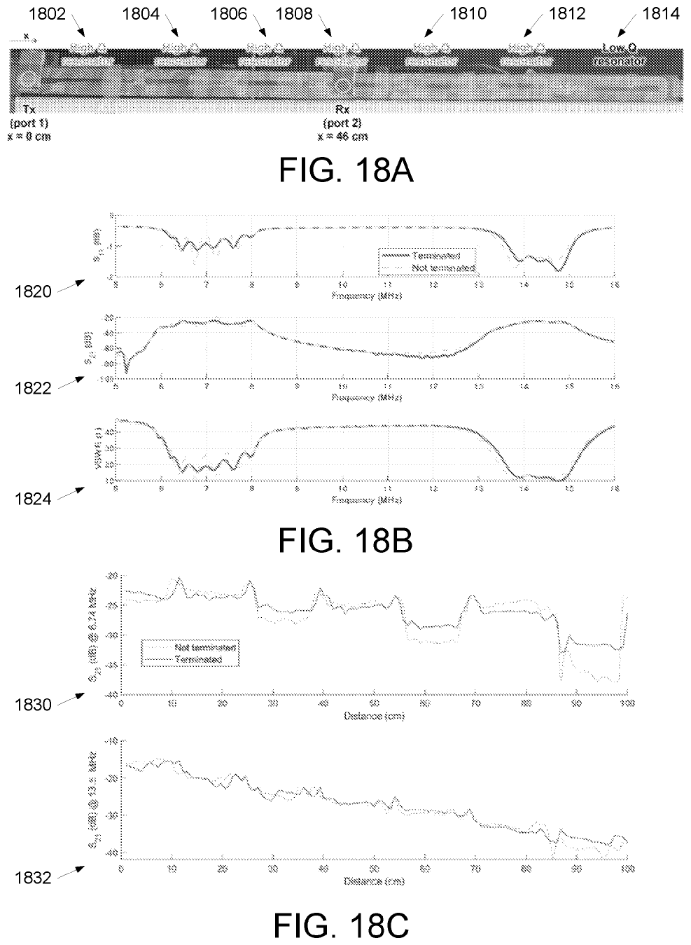

Diagrams illustrating termination of magneto-inductive arrays using low quality resonators at the array's end in accordance with an embodiment of the invention are shown in FIGS. 18a-c. The quality factor in each passband may be lowered using lumped resistors in series with the tuning capacitors. In reference to FIG. 18a, a skyrmion network of 6 resonators (high Q resonators 1802, 1804, 1806, 1808, 1810, 1812) may be terminated with another resonator (low Q resonator 1814) with added lumped resistors. In reference to FIG. 18b, the reflection coefficient (graph 1820), transmission (graph 1822) and voltage standing wave ratio (VSWR) (graph 184) may be monitored with less fluctuations for when the 7th resonator is of low-quality factor (terminated) compared to normal quality factor (unterminated). In reference to FIG. 18c, the transmission profiles versus a moving receiver shows lower transmission fluctuation for the terminated array at both passbands. Specifically, graph 1830 and graph 1832 show the transmission profile for the terminated array at 6.74 MHz and 13.5 MHz, respectively.

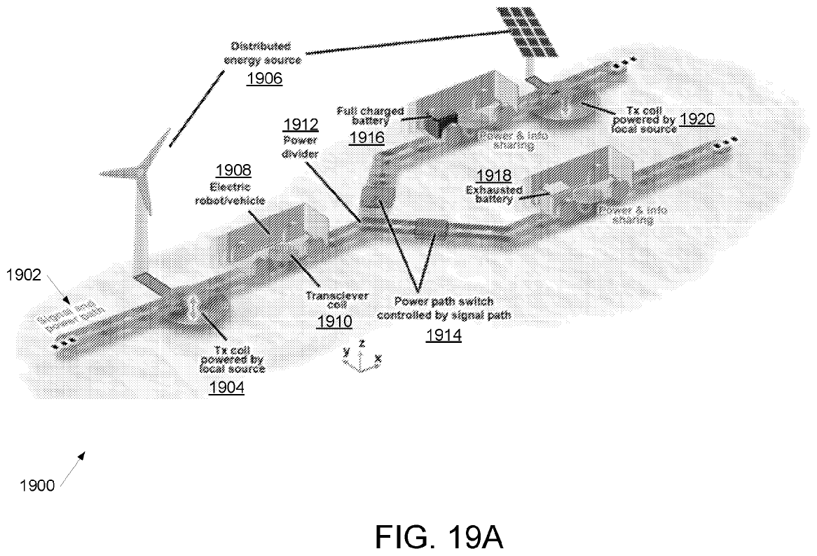

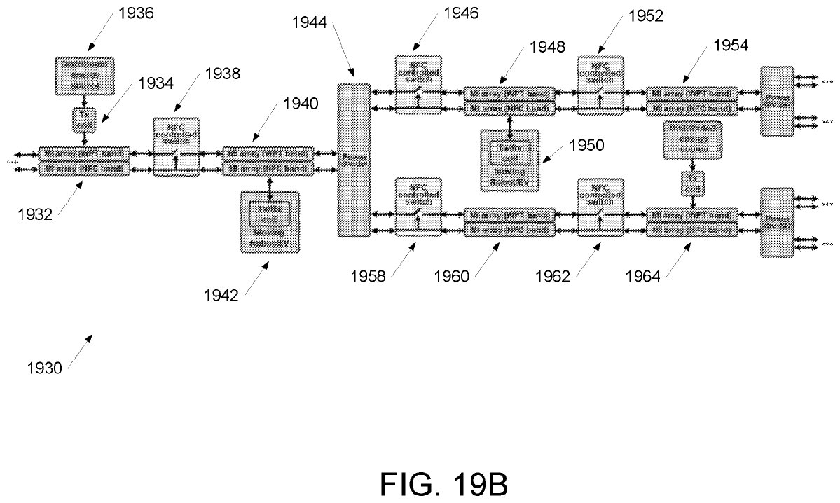

Diagrams illustrating a tunable multiband skyrmion array of magneto-inductive metamaterials can create a low-loss network between several distributed energy sources and electric robot/vehicles close to the network in accordance with an embodiment of the invention are shown in FIGS. 19a-b. In reference to FIG. 19a, by eliminating extra infrastructure to transmit and store the produced renewable energy, this network 1900 maximally adapts to geographical power characteristics of a local climate. For example, the network 1900 may include a signal and power path 1902 connected to one or more distributed energy source(s) 1906. The network 1900 may also include one or more Tx coil(s) 1904, 1920 powered by a local source such as, but not limited to the distributed energy source(s) 1906. As illustrated, an electric robot/vehicle 1908 that includes a transceiver coil 1910 may operate along the signal power path 1902. In some embodiments, the signal and power path 1902 may include a power divider 1912 that allows the signal and power path 1902 to branch into various paths. As illustrated, a first path shows a fully charged battery 1916 of an electric robot/vehicle and a second path shows an exhausted battery 1918 of an electric robot/vehicle. The network 1900 may be configured for power and information sharing, as further described above.

In reference to FIG. 19b, the power safety may be managed through the switches along the magneto-inductive array that communicate through the same network (through NFC) to switch off power transmission in case a failure is detected. The distance between the energy sources can be increased by optimizing the magneto-inductive waveguide dispersion properties. For example, the network 1930 illustrates the management of the power safety of the network 1900 illustrated in FIG. 19A. In many embodiments, the signal and power path may include MI arrays 1932 (WPT band and NFC band) connected to a Tx coil 1934 that is connected to a distributed energy source 1936. The network 1930 may also include an NFC controlled switch 1938 that connects the MI arrays 1932 and MI arrays 1940. As illustrated, various moving electric robot/vehicle 1942 that includes a transceiver (Tx/Rx) coil may operate along the network 1930. In some embodiments, the network 1930 may include a power divider 1944 that allows the signal and power path of the network 1930 to branch into various paths. For example, a first path may include various MI arrays 1948, 1954 and NFC controlled switches 1946, 1952. As illustrated, another moving electric robot/vehicle 1950 that includes a transceiver (Tx/Rx) is shown down the first path. Further, a second path may include various MI arrays 1960, 1964 and NFC controlled switches 1958, 1962.

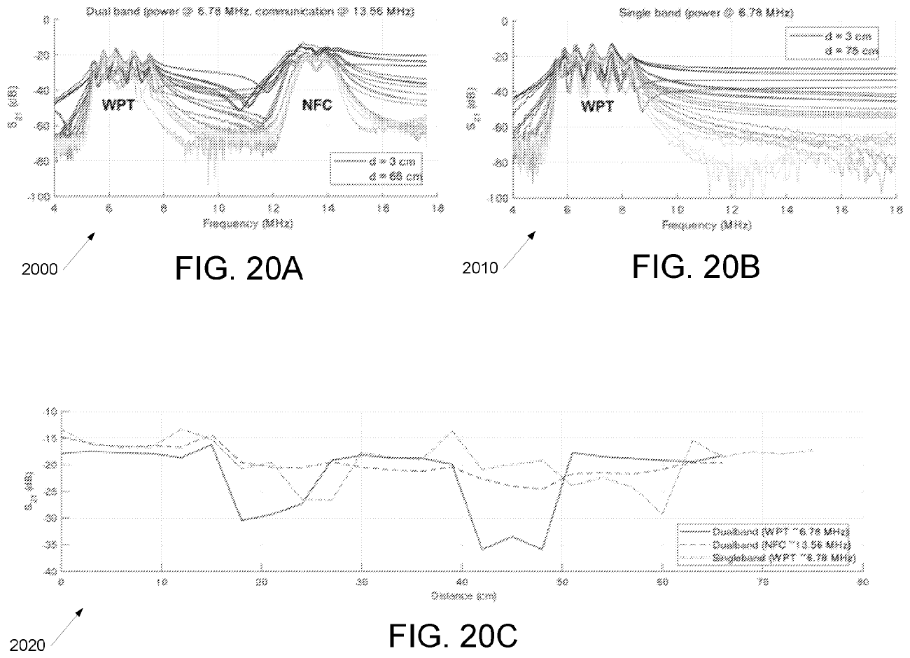

Diagrams illustrating transmission profile of a dual band skyrmion array of magneto-inductive metamaterials in accordance with an embodiment of the invention are shown in FIGS. 20a-c. Transmission profile of a dual band skyrmion array of magneto-inductive metamaterials at both power and communication frequency bands is shown in graph 2000 of FIG. 20a. In reference to FIG. 20b, its comparison to a single band magneto-inductive array is shown in graph 2010. In reference to FIG. 20c, the loss versus distance between the Tx and Rx nodes in each frequency band/scenario is illustrated in graph 2020.

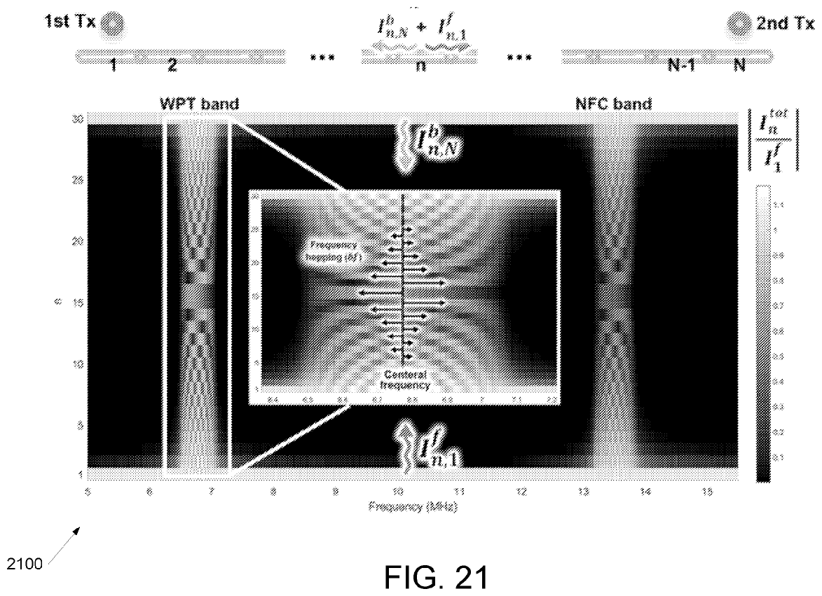

A diagram 2100 illustrating transmission profile fluctuations (demonstrated by normalized current of the n^{th} resonator) caused by multiple sources (above the 1^{st} and 30^{th} resonators) along a terminated magneto-inductive waveguide of 30 resonators in accordance with an embodiment of the invention is shown in FIG. 21. The traveling waves (propagating in different directions between the sources) collide into a standing wave which can cause transmission losses at different frequencies depending on the resonator location. The effect of standing waves can be minimized by implementing a frequency hopping technique.

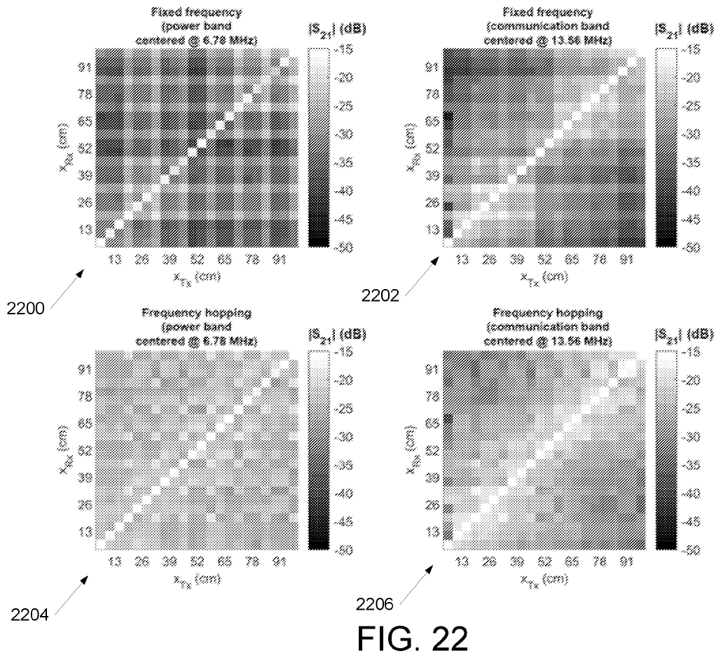

Diagrams illustrating effect of transmission fluctuation versus relative position of Tx and Rx nodes (caused by unterminated magneto-inductive arrays) in accordance with an embodiment of the invention is shown in FIG. 22. Fixed frequency at power bands centered at 6.78 MHz and at 13.56 MHz are shown in diagrams 2200, 2202, respectively. Frequency hopping at power bands centered at 6.78 MHz and at 13.56 MHz are shown in diagrams 2204, 2206, respectively. Minor in-band frequency hopping managed by the transmitter node allows the magneto-inductive network to reach its maximum transmission in each passband.

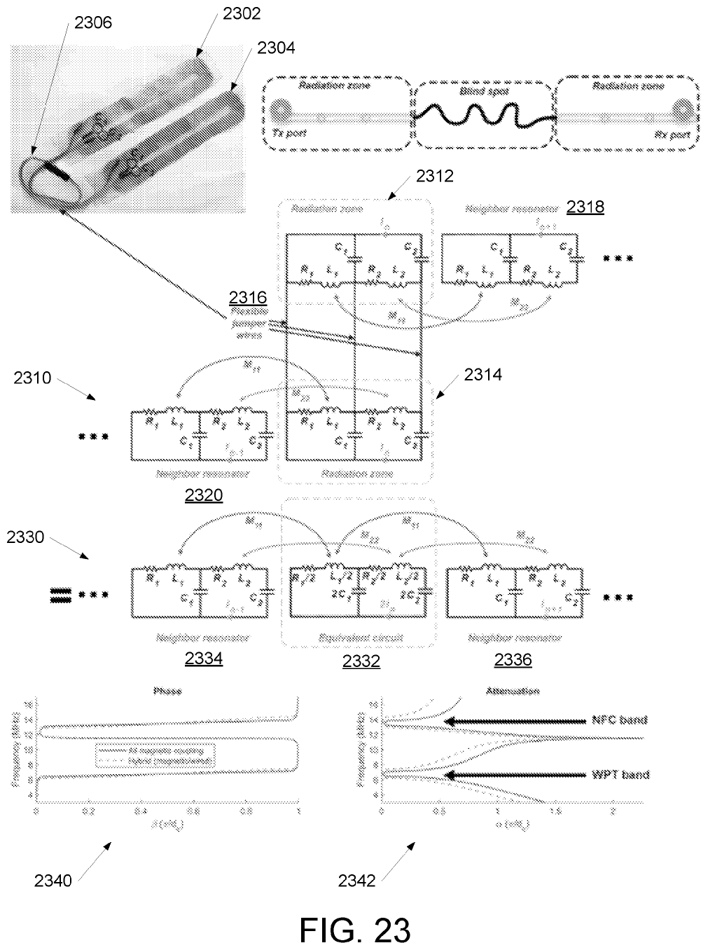

A diagram illustrating hybrid wired connection between two neighbor resonators in a dual band magneto-inductive array tuned at 6.78 MHz and 13.56 MHz for multipurpose powering and communication applications in accordance with an embodiment of the invention is shown in FIG. 23. For example, a hybrid connection may include a first resonator 2302 and a second resonator 2304 with a hybrid wired connection 2306. In reference to FIG. 23, a circuit representation 2310 of the hybrid wired connection is provided. The circuit representation 2310 may include a first radiation zone 2312 and a second radiation zone 2314, inter-connected using flexible jumper wires 2316. In some embodiments, the first radiation zone 2312 may be in proximity to a neighbor resonator 2318 and/or the second radiation zone 2314 may be in proximity to a neighbor resonator 2320. In further reference to FIG. 23, an equivalent circuit representation 2330 of the hybrid wired connection is provided. As illustrated, the equivalent circuit representation 2330 includes an equivalent circuit 2332 in proximity to the neighbor resonators 2334, 2336. Similar to the single band network, the jumper wires create a radiation-free blind spot that enhances the security, horizontal range, and allows for simple switching between two sides of the same array. Graphs 2340, 2342 illustrating the phase and attenuation characteristics, respectively, are also illustrated.

Although specific multi-band skyrmion arrays of magneto-inductive resonators are discussed above with respect to FIGS. 15-23, any of a variety of multi-band skyrmion arrays of magneto-inductive resonators as appropriate to the requirements of a specific application can be utilized in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A tunable multiband skyrmion network comprising:
at least one magneto-inductive “MI” array, wherein the at least one MI array comprises a plurality of magnetically coupled resonators configured to propagate MI surface waves;
at least one transmitter coil powered by a local energy source;
at least one transceiver coil connected to at least one peripheral configured to travel along the at least one MI array; and
wherein the tunable multiband skyrmion network includes power sharing using a power frequency band and data sharing using a communication frequency band.

16. The tunable multiband skyrmion network of claim 15, wherein the power frequency band is a wireless power transfer “WPT” frequency band.

17. The tunable multiband skyrmion network of claim 15, wherein the communication frequency band is a nearfield communication “NFC” frequency band.

18. The tunable multiband skyrmion network of claim 15, wherein the at least one peripheral is a device having a rechargeable battery.

19. The tunable multiband skyrmion network of claim 15 further comprising a power divider and plurality of power path switches controlled by a signal path.

20. The tunable multiband skyrmion network of claim 19, wherein power safety is managed though the plurality of power path switches, wherein each of the plurality of power path switches is configured to switch off power transmission in case of detected failure.

21. The tunable multiband skyrmion network of claim 15, wherein the at least one MI array comprises at least two resonators connected via jumper wires to create a radiation-free blind spot to enhance security, horizontal range, and switching between two side of the at least one MI array.

22. The tunable multiband skyrmion network of claim 15, wherein the data sharing is generated by the plurality of magnetically coupled resonators that create a magnetic metamaterial path for wireless communication using the MI surface waves.

23. The tunable multiband skyrmion network of claim 15, wherein the at least one MI array comprises a plurality of MI arrays, and wherein:
each of the plurality of MI arrays comprises magnetically coupled resonators configured to propagate the MI surface waves;
the magnetically coupled resonators creates a magnetic metamaterial path for the data sharing using the MI surface waves; and
the plurality of MI arrays operates at a plurality of resonance modes.

24. The tunable multiband skyrmion network of claim 23, wherein the plurality of resonance modes comprises a first resonance mode, a second resonance mode, and a third resonance mode.

25. The tunable multiband skyrmion network of claim 24, wherein each resonance mode of the first, second, and third resonance modes, includes induced currents that disorients locally generated magnetic fields.

26. The tunable multiband skyrmion network of claim 25 further comprising at least one capacitor, wherein each resonant mode's frequency is tuned using the at least one lumped capacitor.

27. The tunable multiband skyrmion network of claim 26, wherein the tunable multiband skyrmion network is a multiturn loop trace and at least one lumped capacitor is integrated on various positions on the multiturn loop trace.

28. The tunable multiband skyrmion network of claim 25 further comprising an NFC controlled switch that connects a first MI arrays and a second MI arrays of the plurality of MI arrays.

29. The tunable multiband skyrmion network of claim 23, wherein the power frequency band includes a first power band and a second power band.

30. The tunable multiband skyrmion network of claim 29, wherein the transmitter coil manages in-band frequency hopping allowing the tunable multiband skyrmion network to reach optimal transmission in the first and second power bands.

31. The tunable multiband skyrmion network of claim 30, wherein the first power band is centered at 6.78 MHz and the second power band is centered at 13.56 MHz.

32. The tunable multiband skyrmion network of claim 15, wherein the at least one peripheral is an electric robot.

33. The tunable multiband skyrmion network of claim 15, wherein the at least one peripheral is an electric vehicle.

34. The tunable multiband skyrmion network of claim 15, wherein the local energy source is part of a distributed energy source.
